(19) United States
(12) Patent Application Publication
(10) Pub. No.: US 2012/0195923 A1
(43) Pub. Date: Aug. 2, 2012

(54) USE OF SKIN CARE COMPOSITIONS COMPRISING LAMINARIACEA EXTRACT FOR TREATMENT OF SKIN AGING SIGNS

(75) Inventors: Sylvie L. Turgeon, L'ancienne-Lorette (CA); Laurie-eve Rioux, Saint-Nicolas (CA); Martin Beaulieu, Rimouski (CA)

(73) Assignee: UNIVERSITE LAVAL, QUEBEC, QC (CA)

(21) Appl. No.: 13/377,310

(22) PCT Filed: Jun. 17, 2010
(86) PCT No.: PCT/CA10/00894

§ 371 Date: Apr. 23, 2012

Related U.S. Application Data
(60) Provisional application No. 61/187,877, filed on Jun. 17, 2009.

(51) Int. Cl. A61K 8/73; A61P 17/00; A61Q 19/00; A61Q 19/08; A61K 31/716; A61K 8/97

(52) U.S. Cl. 424/195.17

(57) ABSTRACT

This invention is directed to a skin care composition comprising a laminriacea (brown seaweed) extract and the use of said composition in the treatment of skin aging signs Specifically, it was discovered that a crude extract of the laminariacea Saccharina Longicruris has a strong potential for anti-senescence and anti-cellular aging effect and is efficient to restore the contraction of the collagen network of aged cell lattices to the level of contraction of young cells.

### CROSS REFERENCE TO RELATED APPLICATIONS

This application is a PCT application filed on Jun. 17, 2010 and published in English under PCT Article 21(2), which claims benefit of U.S. provisional application Ser. No. 61/187,877, filed on Jun. 17, 2009. All documents above are incorporated herein in their entirety by reference.

### FIELD OF THE INVENTION

The present invention generally relates to cosmetical, pharmaceutical and nutraceutical compositions and methods of use thereof. The present invention also relates to the prevention and/or treatment of skin conditions and disorders, such as skin-aging signs.

### BACKGROUND OF THE INVENTION

Aging of the skin is a complex biological process which is mediated by a combination of the effects of time (intrinsic aging) and environmental factors (extrinsic aging) on cellular and extracellular infrastructure.

As a result of skin aging, deep changes happen at both dermis and epidermis levels. The amount of water held by the epidermis decreases, skin keratinocytes renewal rate slows down, glycosaminoglycans (GAGs) production decreases, renewal of collagen fibres diminishes and the extracellular matrix (ECM) becomes disorganized. All those events lead to the typical apparition of wrinkles and fine lines, thinning of the skin and loss of firmness, elasticity and moisture.

Over time, dead skin cells do not shed as quickly and turnover of new skin cells may decrease slightly. The skin cells in the bottom layer of the epidermis (stratum basal) constantly split through cell division, forming new keratinocytes. This regenerative process is called skin cell renewal. As we age, the rate of skin cell renewal decreases, causing cells to become stickier and to not shed as easily. As a result of cell renewal decreasing, the skin becomes thinner and more susceptible to environmental damages. Eventually, the skin appears dull and rough in texture.

With age, the synthesis of GAGs decreases, affecting moisture levels in the dermis, collagen synthesis and cellular proliferation. Glycosaminoglycans are associated with proteins to form proteoglycans molecules. In the dermis, proteoglycans interact with collagen fibres allowing their optimal orientation, good dermis structure stability and skin firmness.

Within the skin, the structural protein called collagen which is found in the dermis provides a mesh-like framework of support and strength for the skin. As we age, collagen production decreases and collagen fibres degrade at a faster rate than in earlier years. This results in an overall decrease in the amount of collagen in the dermis. Areas with less support begin to cave in and wrinkles begin to form. Thus, collagen becomes less soluble, thinner, and sparser in intrinsically aged skin. The ratio of type III to type I collagen is reported to increase with age. Histologically, young collagen is randomly organized into a meshwork of loosely interwoven bundles. Aging leads to a loosening within these bundles and straightening of collagen fibres, increasing the skin's tensile strength. Elastin is a long-lived protein in human skin; it appears to accumulate damage with age and sun exposure. New elastin is synthesized in greater quantities in aged skin, but it is thought that this synthesis results in abnormally structured elastin. Also, elastin degradation does not appear to keep pace with new synthesis in aged skin. This results in massive accumulations of elastic material, especially in photo aged skin. The abnormal structure of this elastin prevents it from functioning as it does in young skin.

Healthy human cells are mortal because they can divide only a finite number of times, growing older each time they divide. Thus cells in an elderly person are much older than cells in an enfant. It is believed that shortened telomeres in mitotic cells may be responsible for some of the changes associated with normal aging. Telomeres are specialized DNA-protein complex at the ends of linear chromosomes and consist of the same sequence (TTAGGG) repeated several times. Telomeres allow chromosomal stability, protect chromosome ends against cellular exonucleases and non-homologous end-joining (NHEJ), and distinguish chromosome ends from DNA breaks (De Boeck et al. 2009). During chromosome duplication, the leading strand replicates in the 5′ to 3′ direction smoothly to the end of its template. However, lagging strand synthesis is unable to copy the parental strand completely, inevitably leaving the daughter strand shorter than the parental strand. All following replications results in a gradual loss of DNA at the chromosome end. The very end of the telomere is formed with a single-stranded 3′ containing the TTAGGG which is called the G-overhang. This overhang can loop back and invade homologous double-stranded telomeric tracts to form a large lasso-like structure called t-loop which would cap the end of the telomere (Verdun and Karlseder 2007) via TRF1 gene.

When telomeres become critically too short, the cells cycle arrest and the cells enter apoptosis (Hao et al. 2004) which might be a consequence of aging (Aubert and Lansdorp 2008). Certain types of cells can bypass telomere shortening by producing telomerases that would add the TTAGGG sequence on the leading strand (De Boeck et al. 2009). Only certain stem cells or germ line populations are telomerase positive which have long or indefinite life-spans. Telomerase-reverse transcriptase (TERT) and telomerase RNA component (TERC) are known to extend life span of the cells and prevent senescence (Bodnar et al. 1998, Lin et al. 2008). TERC serves as a template for the TTAGGG synthesis and TERT utilises the integrated RNA molecule for DNA synthesis (De Boeck et al. 2009). Also, the expression telomerase does not seem to be oncogenic (Bodnar et al. 1998).

It has been proposed that telomere shortening may be a molecular clock mechanism that counts the number of times a cell has divided and when telomeres are short, cellular senescence occurs. Once the telomere is reduced to a certain level, the cell can no longer divide. Its metabolism slows down, it ages, and dies.

Despite the number of solutions that have been proposed for reducing signs of aging and stimulating skin renewal, there remains a need for new skin care compositions and methods of use thereof.

The present description refers to a number of documents, the content of which is herein incorporated by reference in their entirety.

### SUMMARY OF THE INVENTION

The present inventors have surprisingly discovered that a laminariacea extract has anti-aging properties on human skin cells. It was also discovered that a subfraction of the laminariacea extract (i.e. the purified laminaran subfraction (e.g., CAS 9008-22-4) has enhanced anti-senescence properties and increases life span of human skin cells.

Accordingly, in a first aspect, the present invention provides a skin care composition comprising a Laminariacea extract. In a specific embodiment, the composition further comprises a topically acceptable carrier. In another specific embodiment, the laminariacea is Saccharina Longicruris. In another specific embodiment, the laminariacea is Laminaria digitata. In another specific embodiment, the extract is a crude Laminariacea extract. In another specific embodiment, the extract is a purified laminaran extract. In another specific embodiment, the extract is a water extract. In another specific embodiment, the purified extract is obtained following a first water extraction followed by an ethanol extraction. In another specific embodiment, the extract is substantially free of fucoidan. In another specific embodiment, the extract is substantially free of polyphenols.

In another specific embodiment, the extract is substantially free of molecules above about 10 kDa. In another specific embodiment, the extract is substantially free of molecules below about 1 kDa. In another specific embodiment, the extract is present at a concentration between about 0.01% and about 5% w/w of the composition. In another specific embodiment, the extract is present at a concentration between about 1.25 to about 5 mg/ml of the composition. In another specific embodiment, the extract is freeze-dried.

In other specific embodiments, Laminariacae extracts of the invention (e.g., S. longicruris extracts) have specific physical characteristics. Hence, in another specific embodiment, the extract comprises laminaran molecules having a molecular weight of about 2000 to 9000 Da. In another specific embodiment, the extract comprises laminaran molecules having a molecular weight of about 2000 to about 6000 Da. In another specific embodiment, the extract comprises laminaran molecules having a size between about 2000 m/z and about 9000 m/z. In another specific embodiment, the extract comprises laminaran molecules having a size between about 4100 m/z and about 5800 m/z. In another specific embodiment, the laminaran extract comprises about 68% of D-glucose and about 1.2% of D-mannitol. In another specific embodiment, the laminaran extract comprises no more than one branching per chain of laminaran. In another specific embodiment, the branching comprises O-6 branching and O-2 branching. In another specific embodiment, the O-6 branching and O-2 branching are in a proportion of about 2% and about 5%, respectively. In another specific embodiment, the laminaran extract comprises laminaran terminated by D-mannitol and laminaran terminated by D-glucose residues. In another specific embodiment, the laminaran terminated by D-mannitol residues and laminaran terminated by D-glucose residues are present in a ratio of 1:1 respectively.

In other specific embodiments, Laminariacae extracts of the present invention (e.g., S. longicruris extracts) have specific properties. Hence, in a specific embodiment, the extract or composition comprising same (a) modulates the expression of a gene involved in cellular senescence; (b) modulates the expression of one or more gene(s) involved in extra cellular matrix (ECM) structure; (c) modulates the expression of one or more gene(s) involved in apoptosis; (d) modulates the expression of one or more gene(s) involved in anti-radical defences; (e) modulates the expression of one or more gene(s) involved in anti-radical differentiation; (f) improves collagen fibres thickness; (g) increases collagen tensing effect; (h) increases the expression of collagen type I; (i) increases cellular energy; a) increases cellular differentiation; (k) increases transglutaminase K activity; (l) reduces crow's feet depth; (m) reduces mouth wrinkles; (n) increases skin firmness; (o) increases skin moisture; (p) improves skin texture; (q) improves skin microrelief; (r) promotes skin repair functions; (s) increases fibroblasts growth; (t) increases the expression of one or more gene(s) involved in telomerase activity; and/or (u) increases telomerase activity. In another specific embodiment, the extract or composition comprising same (a) modulates the expression of one or more gene(s) involved in ECM structure; (b) modulates the expression of one or more gene(s) involved in apoptosis; (c) modulates the expression of one or more gene(s) involved in anti-radical defences; (d) increases collagen tensing effect; (e) increases cellular energy; (f) increases cellular differentiation; (g) increases fibroblasts growth; (h) increases the expression of one or more gene(s) involved in telomerase activity; and/or (i) increases telomerase activity. In another specific embodiment, the extract or composition comprising same (a) increases the expression of at least one of BCL2-like 1, Colony stimulating factor 2, Cyclin F, Dermapontin, Fibromodulin, Heat shock 70 kDa protein 4, Insulin-like growth factor binding protein 5, Intercellular adhesion molecule 1, interleukin 11, Interleukin 8, Matrix metalloprotease 14 (MMP14), Neuregulin 1, Neurofibromin 1, osteopontin, cornifin, mitochondrial Superoxide Dismutase 2, gamma synuclein, Telomerase RNA component, Telomerase reverse transcriptase, Thrombospondin 1, Thromboxane A2 receptor, Transcription factor AP-2 beta, Tumor necrosis factor receptor superfamily member 16 and Tumor protein p53; and/or (b) decreases the expression of at least one of Keratin 10 and Keratin 19. In another specific embodiment, the extract or composition comprising same prevents senescence. In another specific embodiment, the extract or composition comprising same extends the life span of cells. In another specific embodiment, the extract or composition comprising same enhances collagen tensing effect. In another specific embodiment, the extract or composition comprising same modulates apoptosis, increases anti-radical defences, increases anti-radical differentiation and/or improves the extracellular matrix of skin cells. In another specific embodiment, the extract or composition comprising same increases collagen Type-I secretion. In another specific embodiment, the extract or composition comprising same reduces the expression of one or more gene(s) involved in apoptosis, and wherein the one or more gene(s) involved in apoptosis are at least one of BCL2, survivin and TRAIL. In another specific embodiment, the extract or composition comprising same increases the expression of one or more gene(s) involved in anti-radical defences, and wherein the one or more gene(s) involved in anti-radical defences are at least one of glutathion peroxidase, glutathion reductase, heme oxygenase and catalase. In another specific embodiment, the extract or composition comprising same (a) increase the expression of one or more gene(s) involved in extra cellular matrix (ECM) structure, and wherein the one or more gene(s) involved in (ECM) optimization properties are at least one of collagen, decorin, biglycan, fibrillin, tenascin, integrin β1 and plasminogen activator inhibitor 2 (PAI 2), or (b) decreases the expression of one or more gene(s) involved in extra cellular matrix (ECM) structure, and wherein the one or more gene(s) involved in (ECM) optimization properties are at least one of metalloproteinase 1 (MMP1) and metalloproteinase 3 (MMP3). In another specific embodiment, the extract or composition comprising same increases the expression of one or more gene(s) involved in anti-radical differentiation, and wherein the one or more gene(s) involved in anti-radical differentiation includes heat shock protein (HSP27). In another specific embodiment, the extract or composition comprising same increases the expression of at least one of Telomerase RNA Component and Telomerase Reverse Transcriptase.

In accordance with another aspect of the present invention, there is provided an extract of the present invention as described above or a composition comprising this extract as described above, for preventing or reducing a skin condition or disorder in a subject. In a specific embodiment, the skin condition or disorder is a skin aging sign. In another specific embodiment, the prevention or reduction of the skin condition or disorder comprises: (a) modulation of the expression of a gene involved in cellular senescence; (b) modulation of the expression of one or more gene(s) involved in extra cellular matrix (ECM) structure; (c) modulation of the expression of one or more gene(s) involved in apoptosis; (d) modulation of the expression of one or more gene(s) involved in anti-radical defences; (e) modulation of the expression of one or more gene(s) involved in anti-radical differentiation; (f) improvement of collagen fibres thickness; (g) increase of collagen tensing effect; (h) increase of the expression of collagen type I; (i) increase of cellular energy; (j) increase of cellular differentiation; (k) increase of transglutaminase K activity; (l) reduction of crow's feet depth; (m) reduction of mouth wrinkles; (n) increase of skin firmness; (o) increase of skin moisture; (p) improvement of skin texture; (q) improvement of skin microrelief; (r) promotion of skin repair functions; (s) increase of fibroblasts growth; (t) increase of the expression of one or more gene(s) involved in telomerase activity; and/or (u) increase of telomerase activity. In a specific embodiment, In a specific embodiment, the prevention or reduction of the skin condition or disorder comprises: (a) modulation of the expression of one or more gene(s) involved in ECM structure; (b) modulation of the expression of one or more gene(s) involved in apoptosis; (c) modulation of the expression of one or more gene(s) involved in anti-radical defences; (d) increase of collagen tensing effect; (e) increase of cellular energy; (f) increase of cellular differentiation; (g) increase of fibroblasts growth; (h) increase of the expression of one or more gene(s) involved in telomerase activity; and/or (i) increase of telomerase activity.

In another specific embodiment, the prevention or reduction of the skin condition or disorder comprises: (a) increase of the expression of at least one of BCL2-like 1, Colony stimulating factor 2, Cyclin F, Dermapontin, Fibromodulin, Heat shock 70 kDa protein 4, Insulin-like growth factor binding protein 5, Intercellular adhesion molecule 1, interleukin 11, Interleukin 8, Matrix metalloprotease 14 (MMP14), Neuregulin 1, Neurofibromin 1, osteopontin, cornifin, mitochondrial Superoxide Dismutase 2, gamma synuclein, Telomerase RNA component, Telomerase reverse transcriptase, Thrombospondin 1, Thromboxane A2 receptor, Transcription factor AP-2 beta, Tumor necrosis factor receptor superfamily member 1B and Tumor protein p53; and/or b) decrease of the expression of at least one of Keratin 10 and Keratin 19.

In another specific embodiment, the prevention or reduction of the skin condition or disorder comprises a prevention of fibroblast senescence. In another specific embodiment, the prevention or reduction of the skin condition or disorder comprises an extension of the life span of cells. In a specific embodiment, the prevention or reduction of the skin condition or disorder comprises an enhancement of the collagen tensing effect. In a specific embodiment, the prevention or reduction of the skin condition or disorder comprises a modulation of apoptosis, an increase of anti-radical defences, an increase of anti-radical differentiation and/or an improvement of the extracellular matrix of skin cells. In a specific embodiment, the prevention or reduction of the skin condition or disorder comprises an increase of collagen Type-I secretion. In a specific embodiment, the prevention or reduction of the skin condition or disorder comprises a reduction in the expression of one or more gene(s) involved in apoptosis, wherein the one or more gene(s) involved in apoptosis are at least one of BCL2, survivin and TRAIL. In a specific embodiment, the prevention or reduction of the skin condition or disorder comprises an increase in the expression of one or more gene(s) involved in anti-radical defences, wherein the one or more gene(s) involved in anti-radical defences are at least one of glutathion peroxidase, glutathion reductase, heme oxygenase and catalase.

In another specific embodiment, the prevention or reduction of the skin condition or disorder comprises an increase in the expression of one or more gene(s) involved in extra cellular matrix (ECM) structure, and wherein the one or more gene(s) involved in (ECM) optimization properties are at least one of collagen, decorin, biglycan, fibrillin, tenascin, integrin β1 and plasminogen activator inhibitor 2 (PAI 2), or (b) decreases the expression of one or more gene(s) involved in extra cellular matrix (ECM) structure, and wherein the one or more gene(s) involved in (ECM) optimization properties are at least one of metalloproteinase 1 (MMP1) and metalloproteinase 3 (MMP3).

In another specific embodiment, the prevention or reduction of the skin condition or disorder comprises an increase in the expression of one or more gene(s) involved in anti-radical differentiation, and wherein the one or more gene(s) involved in anti-radical differentiation includes heat shock protein (HSP27). In another specific embodiment, the prevention or reduction of the skin condition or disorder comprises an increase in the expression of at least one of Telomerase RNA Component and Telomerase Reverse Transcriptase.

In accordance with yet another aspect of the present invention, there is provided a method for preventing or reducing a skin condition or disorder in a subject, comprising administering an effective amount of the extract of the present invention as described above or of a composition comprising this extract and a topically acceptable carrier, on the subject's skin.

In a specific embodiment, the skin condition or disorder is a skin aging sign.

In accordance with yet another aspect of the present invention, there is provided a use of the extract as described above or of a composition comprising this extract and a topically acceptable carrier, for the manufacture of a medicament.

In accordance with yet another aspect of the present invention, there is provided a use of the extract as described above or of a composition comprising this extract and a topically acceptable carrier, for the manufacture of a medicament for preventing or reducing a skin condition or disorder.

In accordance with yet another aspect of the present invention, there is provided a use of the extract as described above or of a composition comprising this extract and a topically acceptable carrier, for preventing or reducing a skin condition or disorder.

In a specific embodiment, the skin condition or disorder is a skin aging sign.

In accordance with yet another aspect of the present invention, there is provided a pharmaceutical and/or nutraceutical and/or dietary composition comprising the extract defined above, and carrier (e.g., a pharmaceutical and/or nutraceutical and/or dietary acceptable).

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows a method of preparing a crude extract of Saccharina longicruris in accordance with the present invention. The final concentration of the extract is 7 mg/ml;

FIG. 2 shows the effect of crude extracts (1.5% w/v and 3% w/v cell media) of Saccharina longicruris on cellular metabolism (cellular respiration) of a primary culture of fibroblasts grown in monolayers;

FIG. 3 shows the effect of a crude extract of Saccharina longicruris (0.08% w/v) on cellular contraction in old cells (passage 14);

FIG. 4 shows the effect of a crude extract of Saccharina longicruris (0.75% w/v) on cellular differentiation;

FIG. 5 shows the results of a consumer study on the effect of a skin care composition comprising 3% w/v of a crude extract of Saccharina longicruris on skin appearance and signs of aging;

FIG. 6 shows the results of stability tests of a solution comprising 30% w/v of crude extract Saccharina longicruris in glycerol A) at 50° C. (upper panel); and B) at room temperature (lower panel);

FIG. 7 shows the electrospray ionisation mass spectra of purified laminaran;

FIG. 8 shows (A) the composition in monosaccharide and molecular weight of purified laminaran and (B) the analysis of methylated reduced laminaran alditol acetates from purified laminaran;

FIG. 9 shows the effect of a purified laminaran extract on the expression of Telomerase RNA Component (TERC) and Telomerase Reverse Transcriptase (TERT);

FIG. 10 shows the effect of a purified laminaran on collagen-I secretion in normal skin dermal fibroblasts. Data are the means±SD carried out in three different experiments. Significant differences between cells treated with fetal bovine serum (FBS) 0.5% (control) and laminaran were calculated using Student test (*P<0.05);

FIG. 11 shows the effect of a purified laminaran on matrix metalloproteinases (MMPs) specific activity in normal skin dermal fibroblasts. Results reflect the overall MMP quantification from the supernatants of cell in culture with purified laminaran at different concentrations (1.25, 2.5 and 5 mg/mL (0.125, 0.25 and 0.5% w/v)). Data are the means±SD carried out in three different experiments. Significant differences between cells treated with FBS 0.5% (control) and purified laminaran were calculated using Student test (*P<0.05 and **P<0.01); and

FIG. 12 shows the effect of commercial crude laminaran extract from Laminaria digitata on telomerase activity.

### DESCRIPTION OF ILLUSTRATIVE EMBODIMENTS

The present invention is illustrated in further details by the following non-limiting examples.

Saccharina longicruris, is a species found in North America and more precisely, in eastern Canada. This seaweed is composed of a long waved frond on the side and is yellow-brown in color. It can measure up to 7 meters (Chapman, 1987). The fronds of S. longicruris contains, on dry weight, 12.2% of proteins, 0.8% of lipids, 26.3% ashes, and per subtraction 60.7% of carbohydrates (Rioux et al, 2009).

Laminaria digitata is a brown alga in the Division Heterokontophyta, of the genus Laminaria, also known by the common name Oarweed. It is also found in North America and is a familiar glossy golden brown kelp, which grows attached to rocky substrates with a dome-shaped cluster of root-like holdfasts. The smooth and flexible stipe (stem) is oval in cross-section, and gives rise to a broad frond that is divided into ribbon-like segments and lacks a midrib.

Both Laminaria digitata and Saccharina longicruris are both brown seaweed of the Laminariacea family.

The present inventors have shown that Saccharina longicruris and Laminaria digitata extracts reverse or prevent the normal aging process of the skin and activates the protection systems of the cells. Thus, the skin care compositions of the present invention prevents or improve a skin condition or disorder in a subject, including skin-aging signs.

In an embodiment, the skin care compositions of the present invention enhance collagen tensing effect. One particular aspect of the invention relates to the ability of the extract to induce the expression of several beneficial genes including telomerase-reverse transcriptase (TERT) and telomerase RNA component (TERC) which are known to extend life span of the cells and prevent senescence.

As used herein the term “crude” refers to a non-fractionated water extract from laminariacea brown seaweed. In a particular embodiment of the present invention, the crude extract is from Saccharina longicruris. In another particular embodiment of the present invention, the crude extract is from Laminaria digitata. A crude extract may be a concoction (blend) of any type, or combination thereof of unfractionated water extraction such as the extraction described in Example 1. The crude extract of Example 1 comprises between 2% and 6% of laminaran.

As used herein the term “purified laminaran extract” refers to a crude extract of laminariacea which has been further purified to increase its content in laminaran. In an embodiment, the purified laminaran extract comprises between 70 and 100% w/w, preferably between 75 and 100% w/w of laminaran, more preferably between 85 and 100% w/w laminaran, more preferably, between 90 and 95% w/w of laminaran, and even more preferably between 95 and 98% w/w of laminaran. In an embodiment, the extract may be filtered or fractionated using molecular sieving or specific fractionation techniques well known in the art. The extract may also be further concentrated or diluted. It can also be lyophilized (freeze-dried) or otherwise treated to increase at least one of its skin care properties. In an embodiment, the extract comprises laminaran molecules having a size between about 2000 m/z and about 9000 m/z. In an embodiment, the extract comprises laminaran molecules having a size between about 4100 m/z and about 5800 m/z. In another embodiment, the extract comprises laminaran molecules having a molecular weight between about 2 kDa and about 9 kDa. In yet a further embodiment, the extract comprises laminaran molecules having a molecular weight between about 2 kDa and about 6 kDa. In a specific embodiment, the extract comprises laminaran molecules having a mean molecular weight of about 2.9 kDa.

Laminaran is the glycosidic reserve of brown seaweed. It is a small glucan with a degree of polymerisation between 20 and 25 (Chizhov, A. O., Dell, A., et al. 1998, Nelson, T. E. and Lewis, B. A. 1974). Laminaran is present in two forms: soluble and insoluble. The first form is characterized by a complete solubility in cold water while the other is only soluble in hot water (Black, W. A. P. and Dewar, E. T. 1973, Percival, E. and McDowell, R. H. 1967). Solubility is also influenced by the presence of branching. The higher the branching content the higher is the solubility in cold water. Two types of laminaran have been described, one type with chains that are terminated by D-mannitol residues (M-series) and the other type with chains terminated by D-glucose residues (G-series) (Nelson, T. E. and Lewis, B. A. 1974). Laminaran is composed of D-glucose linked in b (1,3) (Barry, V. 1939) with intrachain branching linked in b (1,6) (Peat, S., Whelan, W. J., et al. 1958). Ratios of the two types of laminaran as well as their structure vary according to the seaweed species and environmental factor such as nutritive salts and the fronds age (Chizhov, A. O., Dell, A., et al. 1998; Rioux et al., 2008).

In an embodiment, the extract is substantially free of molecules having a molecular weight below 1 kDa (>1 kDa fraction). Such an extract may be obtained, for example, by collecting the retentate fraction following filtration of the above-mentioned extract on a filter having a cut-off of 1 kDa. Other separations means such as electrodialysis, electrofractionating or ethanol precipitation may be utilized. In an embodiment, the extract is substantially free of molecules having a molecular weight above 10 kDa (<10 kDa fraction). Such an extract may be obtained, for example, by collecting the filtrate fraction following filtration of the above-mentioned extract on a filter having a cut-off of 10 kDa. In another embodiment, the extract is substantially free of molecules having a molecular weight above 10 kDa and below 1 kDa (1-10 kDa fraction). Such an extract may be obtained, for example, by collecting the filtrate fraction following filtration of the above-mentioned extract on a filter having a cut-off of 10 kDa, and submitting this filtrate fraction to a second filtration on a filter having a cut-off of 1 kDa, and collecting the retentate fraction. “Substantially free” as used herein, is meant to reflect the inherent imprecision of filtration membranes or other separation equipments.

As used herein, the terminology “w/v” (weight by volume) refers to the weight of the product in g divided by the volume in mL. Therefore the equation is Mass (g)/Volume (mL)×100=%. For example, a 1% solution would have 1 g of solute dissolved in a final volume of 100 mL of solution.

### Types of Formulations of Extracts of the Present Invention

In addition, the present invention provides a pharmaceutical and/or nutraceutical and/or dietary supplement composition comprising an extract of the present invention. The pharmaceutical and/or nutraceutical and/or dietary supplement composition of the present invention can contain a pharmaceutically acceptable carrier for administration to a mammal, including, without limitation, sterile aqueous, or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. Pharmaceutically acceptable carriers also can include physiologically acceptable aqueous vehicles (e.g., physiological saline) or other known carriers appropriate to specific routes of administration.

The compositions of the present invention may be formulated in a topically applicable pharmaceutical or cosmetic composition (e.g., a topical formulation). Carriers included in topically applicable compositions of the present invention are topically acceptable carriers. Non-limitative examples of such topically applicable compositions include skin care cream, cleansing cream, ointment, skin care lotion, skin care gel, skin care foam, sun care composition, make-up removal cream, make-up removal lotion, foundation cream, liquid foundation, bath and shower preparation, deodorant composition, antiperspirant composition, shaving products composition, after-shave gel or lotion, beauty aids composition, depilatory cream, soap composition, hand cleaner composition, cleansing bar, baby care, hair care, shampoo, setting lotion, treatment lotion, hair cream, hair gel, coloring composition, restructuring composition, permanent composition, anti-hair loss composition, or any other composition which is adapted for the use in a topical cosmetic regimen.

Creams, as is well known in the art of pharmaceutical and cosmeceutical formulation, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a non-ionic, anionic, cationic or amphoteric surfactant.

Lotions are preparations to be applied to the skin surface without friction, and are typically liquid or semi liquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution may contain other cosmeceutically acceptable chemicals to buffer, stabilize or preserve the solute. Common examples of solvents used in preparing solutions are ethanol, water, propylene glycol or any other cosmeceutically acceptable vehicles.

Gels are semisolid, suspension-type systems. Single-phase gels contain organic macromolecules distributed substantially uniformly throughout the carrier liquid, which is typically aqueous, but also, preferably contain an alcohol, and, optionally, an oil. “Organic macromolecules,” i.e., gelling agents, are cross linked acrylic acid polymers such as the “carbomer” family of polymers, e.g., carboxypolyalkylenes that may be obtained commercially under Carbopol™. The carbomer formulation described in certain Examples herein is a gel. Other examples are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Ointments are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, Pa.: Mack Publishing Co., 1995), at pages 1399-1404, and ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall comprising a lipid bilayer, and, in the present context, encapsulate one or more components of the anti-aging formulations. Liposomal preparations herein include cationic (positively charged), anionic (negatively charged), and neutral preparations. Cationic liposomes are readily available. For example, N[1-2,3-dioleyloxy)propyl]-N,N,N-triethylammonium (DOTMA) liposomes are available under the tradename Lipofectin™ (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl-8 dodecyl ether, polyoxyl-12 dodecyl ether, nonoxynol 10, and nonoxynol 30.

Microspheres, similarly, may be incorporated into the present formulations. Like liposomes and micelles, microspheres essentially encapsulate one or more components of the present formulations. They are generally although not necessarily formed from lipids, preferably charged lipids such as phospholipids. Preparation of lipidic microspheres is well known in the art and described in the pertinent texts and literature.

### Topically/Cosmetically Acceptable Carrier, Vehicle, Excipient or Additives for Extracts of the Present Invention

In an embodiment, the composition of the present invention further comprises a pharmaceutically and/or cosmetically acceptable topical carrier, vehicle, excipient or additives (i.e. topically/cosmetically acceptable carrier, vehicle, excipient or additives). Such carrier, vehicle, excipient or additives are well known in the art and may be used, for example, to improve final formulation regarding organoleptic properties, skin penetration and accessibility of the active ingredient. Examples of carriers, vehicles or excipients include: buffering agent, carrier agent, chelating agent, conditioner agent, coloring agent, detackifier agent, emollient agent, emulsifier agent, film former agent, foaming agent, humectant agent, lactylate agent, lipophilic agent, lubricant agent, neutralizer agent, oil agent, opacifier agent, preservative agent, solubilizer agent, solvent agent, stabilizer agent, surfactant agent, thickener agent, viscosity agent, water absorbent agent, wetting agent, perfume and thermal water. It also includes any combination thereof.

The composition of the present invention may be formulated so as to provide for a specifically controlled delivery system. Non-limitative examples of such delivery systems include slow delivery system, rapid delivery system, immediate delivery system, delayed delivery system, zero-order delivery system and dual or multiple speed delivery system. Such controlled delivery systems may be achieved with specific formulations including chemical delivery systems, multiple emulsions, microemulsions, nanoemulsions, encapsulations such as liposomes, microspheres, nanospheres, microsponges, beads and cyclodextrins, polymeric matrices, polymeric cosmetic conjugates, oil body/oleosin, oil-soluble molecular film, skin patches, unit dosages.

Without being so limited, buffering agents are salts of bases/acids, compatible with the nature of the skin and with its pH. Sodium acetate is an example of a frequently used buffer agent.

Without being so limited, carrier agents are ingredients capable of aiding the application of the active ingredient. Isohexadecane is an example of a frequently used carrier.

Without being so limited, chelating agents are ingredients capable of binding mono and divalent cations, such as EDTA, trisodium EDTA, tetrasodium EDTA, disodium EDTA or a combination thereof.

Without being so limited, conditioner agents are ingredients with lubricating action and hydrating effect, such as cetrimonium chloride, dicetyldimonium chloride, trideceth-12, quaternium-Z7, quaternium-18, polyquatemium-10, behentrimonium methosulfate, cetearyl alcohol, stearamidopropyl dimethylamine, trimethylsilylamodimethicone, isolaureth-6, octoxynol-4, dimethicone, dimethiconol, cyclopentasiloxane, pareth-7, pareth-9, linoleic acid and glycerin, or a combination thereof.

Without being so limited, detackifier agents are ingredients capable of adsorbing onto tacky materials and reduce their tendency to adhere, such as cyclopentasiloxane, dimethicone and vinyl dimethicone, phenyl trimethicone, isopropyl esters, isostearate esters, dimethyl sebacate and dipropyl sebacate, or a combination thereof.

Without being so limited, emollient agents are ingredients with lubricating action and hydrating effect, such as isopropyl palmitate, sunflower seed oil, mineral oil, stearyl stearate, isopropyl myristate, lanolin, caprylic, capric triglyceride, cyclopentasiloxane, dimethicone, vinyl dimethicone, bis-phenylpropyl dimethicone, alkyl dimethicone, sorbitan stearate, sucrose distearate, myristyl alcohol, myristyl lactate, cetyl acetate, dicaprylyl ether, floraester-20, maleated soybean oil, cyclomethicone, shea butter, hydrogenated coconut oil, isopropyl palmitate, diisostearoyl trimethylolpropane siloxy silicate and alkyl benzoate, or a combination thereof.

Without being so limited, emulsifier agents are ingredients capable of preventing the separation of immiscible substances in an emulsion, of helping to distribute evenly one substance in another, of improving texture, homogeneity, consistency and stability, such as cetearyl alcohol, glyceryl stearate, alkyl acrylate crosspolymer, stearic acid, emulsifying wax, sorbitan oleate, sorbitan stearate, polysorbate, polyethylene glycopolysorbate, triethanolamine, cyclopentasiloxane, dimethicone copolyol, PEG-30 dipolyhydroxystearate, sucrose distearate, PEG-100 stearate, sodium dioctylsulfosuccinate, polyacrylamide, isoparaffin, laureth-7, cetyl phosphate, DEA cetyl phosphate, glycol stearate, stearyl alcohol, cetyl alcohol, behentrimonium methosulfate and ceteareth-2, or a combination thereof.

Without being so limited, film former agents are ingredients capable of forming a dimensionally stable and continuous film to minimize the formula tackiness, such as wheat protein, eicosene copolymer, perfluoromethylisopropyl ether, diisostearoyl trimethylolpropane siloxy silicate, trimethylsiloxysilicate, dimethicone, vinyl dimethicone and cyclopentasiloxane, or a combination thereof.

Without being so limited, foaming agents are ingredients capable of regulating the amount of air in a product, such as lauramide DEA and cocamide MEA, disodium laureth sulfosuccinate, disodium N-octadecyl sulfosuccinamate, ammonium lauryl sulphate, triethanolamine lauryl sulfate, sodium lauryl sulphate and sodium 2-ethylhexylsulfate, or a combination thereof.

Without being so limited, humectant agents are ingredients capable of maintaining constant humidity and retaining moisture, such as glycerine, PEG-8, butylene glycol and propylene glycol, or a combination thereof.

Without being so limited, lubricant agents are ingredients capable of adding slipperiness and reducing friction to improve application, such as dimethicone and dimethicone copolyol, or a combination thereof.

Without being so limited, neutralizer agents are ingredients capable of changing the acid-alkaline balance, such as triethanolamine and sodium hydroxide, or a combination thereof.

Without being so limited, opacifier agents are ingredients capable of changing the look of a clear or translucent product to a creamier or pearlier one, such as glyceryl stearate and PEG-100 stearate, or a combination thereof.

Without being so limited, preservative agents are ingredients capable of retarding or preventing microbial or chemical spoilage and protecting against discoloration, such as DMDM hydantoin, methylparaben, propylparaben, phenoxyethanol, ethylparaben, butylparaben, imidazolidinyl urea, diazolidinyl urea, quaternium-8, quaternium-14, quatemium-15, propylene glycol, dehydroacetic acid, methylchloroisothiazolinone, methylisothiazolinone and germaben, or a combination thereof.

Without being so limited, solubilizer agents are ingredients capable of allowing incompatible ingredients to become part of a homogeneous solution, such as polysorbate, ceteareth, steareth and PEG, or a combination thereof.

Without being so limited, stabilizer agents are ingredients capable of maintaining physical and chemical properties during and after processing, preventing or limiting changes in the physical properties of a substance during product life, such as polyethylene, sodium chloride, stearyl alcohol, xanthan gum, tetrasodium EDTA and dimethicone copolyol, or a combination thereof.

Without being so limited, surfactant agents are ingredients capable of reducing surface tension when dissolved in water or a water solution, reducing interfacial tension between two liquids or between a liquid and a solid, such as sodium dioctylsulfosuccinate, octoxynol-40, isolaureth-6, ammonium lauryl sulfate, lauryl alcohol, lauramide DEA and cocoamidopropyl betaine, or a combination thereof.

Without being so limited, thickener agents are ingredients capable of absorbing water to impart body, improve the consistency or texture, and stabilize an emulsion, such as stearic acid, magnesium aluminum silicate, carbomer (including sodium carbomer and potassium carbomer), alkyl acrylate crosspolymer, polyacrylamide, isoparaffin, laureth-7, cetyl alcohol, xanthan gum, alkyl dimethicone, hydroxyethylcellulose, glyceryl stearate, pentaerythrityl tetrastearate, stearyl alcohol and polyquaternium-10, or a combination thereof.

Without being so limited, viscosity agents are ingredients capable of controlling the degree of fluidity and the internal resistance to flow exhibited by a fluid, such as magnesium aluminum silicate, caprylyl glycol and myristyl alcohol, or a combination thereof.

Without being so limited, water absorbent agents are ingredients capable of absorbing the product's water to maintain the moisture, such as carboxyvinyl polymer, acrylic copolymer, polyacrylamide, polysaccharides, natural gum, clay, modified clay, metallic salt, fatty acid, or a combination thereof.

Without being so limited, wetting agents are ingredients capable of reducing the surface tension of the water for better penetration or spread over the surface, such as caprylate, caprylyl glycol, glyceryl caprate, polyglyceryl-2 caprate, polyglyceryl-6, polyglyceryl-3 laurate and TEA-laureth sulfate, or a combination thereof.

### Additional Active Agent

In an embodiment, the composition of the present invention may further comprise at least one additional active agent, such an agent that modulates cell differentiation or proliferation, an anesthesic agent, anti-acne agent, anti-aging agent, antibacterial agent, anticellulite agent, antifungal agent, anti-inflammatory agent, anti-irritant agent, antioxidant agent, antiparasitic agent, antipollution agent, antipruritic agent, anti-rosacea agent, anti-seborrhea agent, anti-stress agent, anti-telangiectasia agent, antiviral agent, anti-wrinkle agent, baby care agent, bath and body agent, calming agent, cleansing agent, collagen synthesis agent, elastase inhibitory agent, exfoliant agent, facial peeling agent, firming agent, foot care agent, free radical scavenging agent, immune function modulator agent, keratolytic agent, lift agent, make-up remover agent, melanogenesis stimulator agent, hair care agent, matrix metalloproteinase inhibitory agent, moisturizing agent, oil absorbent agent, osmoregulator agent, anti-photoaging agent, protecting agent, rejuvenating agent, regenerating agent, restructuring agent, sensitive skin agent, shaving product agent, skin defense enhancer agent, skin clarifier agent, skin repair agent, slimming agent, smoothing agent, softening agent, soothing agent, sun care agent, sunless tanning agent, tensing agents and whitening agent, or any other agent adapted for use in a cosmetic regimen that comprises topical application of the cosmetic composition, and which complements or supplements the effect of the extract composition of the present invention. In a further embodiment, the above-mentioned at least one additional active ingredient modulate(s) at least one of cell differentiation, cell metabolic activity, cell structure, cell proliferation, extracellular processes and pigmentation.

Without being so limited, agents that modulate cell differentiation or proliferation include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), vitamin D and its derivatives (cholecalciferol, ergocalciferol, 25-hydroxycholecalciferol), growth factors, estradiol derivatives. It also includes any combination thereof.

Without being so limited, anaesthesics include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include lidocaine chlorhydrate and its derivatives. It also includes any combination thereof.

Without being so limited anti-acne agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include benzoyl peroxide, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), salicylic acid, sulfur, sulfurated lime, alcohol and acetone. It also includes any combination thereof.

Without being so limited, anti-aging/anti-wrinkle agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include hyaluronic acid, sodium-2-pyrrolidone carboxylate, glycosaminoglycans, kinetin, retinoic acid and its derivatives (retinal, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), epidermal growth factor, ceramide, ethylbisiminomethylguaiacol manganese chloride, glycation inhibitors, chrysanthellum indicum extract and aphanizomenon flos aquae extract. It also includes any combination thereof.

Without being so limited, antibacterial agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include eucalyptus extract, clindamycin phosphate, cavacrol, erythromycin and antibiotics belonging to the group of tetracyclines. It also includes any combination thereof.

Without being so limited, antifungal agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include econazole, ketoconazole, miconazole, amphotericin B, terbinafine and octopirox. It also includes any combination thereof.

Without being so limited, anti-inflammatory agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include allantoin, vitamin E and its derivatives (α-tocopherol, δ-tocopherol, γ-tocopherol), chamomile oil, gingko biloba oil and camellia sinensis extract. It also includes any combination thereof.

Without being so limited, anti-irritant/soothing/smoothing/calming agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include allantoin, camellia sinensis extract, lavender oil, aloe vera, linden extract, epilobium angustifolium extract, chysanthellum indicum extract, cola nitida extract and alteromonas ferment extract. It also includes any combination thereof.

Without being so limited, antioxidant agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include furfuryladenine, panthenol, lipoic acid, ubiquinone, niacinamide, melatonin, catalase, glutathione, superoxide dismutase, polyphenols, cysteine, allantoin, kinetin, vitamin C and its derivatives (ascorbyl palmitate, magnesuim ascorbyl phosphate, sodium ascorbyl phosphate), vitamin E and its derivatives (α-tocopherol, δ-tocopherol, γ-tocopherol), grape seed extract and camellia sinensis extract. It also includes any combination thereof.

Without being so limited, antipruritic agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include thenaldine, trimeprazine, cyproheptadine. It also includes any combination thereof.

Without being so limited, anti-rosacea/anti-telangiectasia agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include metronidazole, vasoconstrictors, benzoyl peroxide, azelaic acid, sulphur, soy proteins and glycosaminoglycans. It also includes any combination thereof.

Without being so limited, anti-seborrhea agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include progesterone derivatives, isoleutrol and hinokitiol. It also includes any combination thereof.

Without being so limited, sensitive skin agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include rose oil and jasmine oil. It also includes any combination thereof.

Without being so limited, cleansing agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include ammonium lauryl sulfate, ammonium laureth sulfate, cocamide MEA, triethanolamine lauryl sulfate, sodium stearate and nettle leaf extract. It also includes any combination thereof.

Without being so limited, collagen synthesis agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), vitamin C and its derivatives (ascorbyl palmitate, magnesium ascorbyl phosphate, sodium ascorbyl phosphate), growth factors and its derivatives. It also includes any combination thereof.

Without being so limited, exfoliant agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include alpha/beta hydroxy acids, salicylic acid, glycolic acid, lactic acid, citrus acid and walnut shell powder. It also includes any combination thereof.

Without being so limited, facial peeling agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include glycolic acid, lactic acid, trichloroacetic acid and phenol. It also includes any combination thereof.

Without being so limited, firming/tensing agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include dimethylaminoethanol, neuro-cosmetic actives (Botox™-like), chitosan, arnica extract, fennel-sweet oil and papaya extract. It also includes any combination thereof.

Without being so limited, free radical scavenging/antipollution/anti-stress agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include grape seed extract, alpha-tocopherol and the esters thereof, superoxide dismutase, some chelating agents of metals, vitamin C and its derivatives (ascorbyl palmitate, magnesium ascorbyl phosphate, sodium ascorbyl phosphate). It also includes any combination thereof.

Without being so limited, hair care agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include poly-D-glucosamine, poly-N-acetyl-D-glucosamine, stearalkonium chloride and triethanolamine lauryl sulfate. It also includes any combination thereof.

Without being so limited, matrix metalloproteinase inhibitory agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include camellia sinensis extract, polyphenols, spatholobi caulis extract, euonymus alatus extract, rhizoma notopterygii extract, quercetin, glycosaminoglycans, polymethoxy flavonoid, N-acetyl-cysteine, 2-furildioxime, isoflavone, vitamin C and its derivatives (ascorbyl palmitate, magnesium ascorbyl phosphate, sodium ascorbyl phosphate), retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, 0-carotene, retinyl ester) and hydroxamate derivatives. It also includes any combination thereof.

Without being so limited, moisturizing agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include cucumber extract, sodium-2-pyrrolidone carboxylate, sodium PCA, sodium hyaluronate, chitin and its derivatives, alpha hydroxy acids, hyaluronic acid and hydrolysed wheat protein. It also includes any combination thereof.

Without being so limited, osmoregulator agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include mannitol, dulcitol and betaine.

Without being so limited, protecting agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include poly-N-acetyl-D-glucosamine, poly-D-glucosamine, alkyloamides, chitosan, chrysanthellum indicum extract, camellia sinensis extract and alteromonas ferment extract. It also includes any combination thereof.

Without being so limited, rejuvenating agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include rosemary extract, rosewood extract, geranium extract and vitamin E and its derivatives α-tocopherol, δ-tocopherol, γ-tocopherol). It also includes any combination thereof.

Without being so limited, skin repair agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), allantoin, eucalyptus extract, lavender oil, rose oil and activators of collagen synthesis and activators of components of the skin's extracellular matrix. It also includes any combination thereof.

Without being so limited, slimming/anticellulite agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include chrysanthellum indicum extract, dihydromyricetin, theobromine, theophylline, aminophylline, caffeine, isopropylarterenol hydrochloride, epinephrine, α-MSH agonists, adenylate cyclase activators and phosphodiesterase inhibitors. It also includes any combination thereof.

Without being so limited, sun care/photo aging agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include PABA (p-aminobenzoic acid) and derivatives, gluconolactone, salicylates, cinnamates, benzophenones, dibenzoylmethanes, oxybenzone, vitamin E and its derivatives (α-tocopherol, δ-tocopherol, γ-tocopherol), ethylbisiminomethylguaiacol manganese chloride, glycosaminoglycans, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), titanium dioxide, octyl methoxycinnamate, benzophenone, octyl salicylate, epilobium angustifolium extract, rumex occidentalis extract, chrysanthellum indicum extract, camellia sinensis extract and alteromonas ferment extract. It also includes any combination thereof.

Without being so limited, sunless tanning/melanogenesis stimulator agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include dihydroxyacetone, α-MSH agonists, adenylate cyclase activators and phosphodiesterase inhibitors. It also includes any combination thereof.

Without being so limited, toning agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include nettle extract, orange blossom extract, rosewood extract and witch hazel extract. It also includes any combination thereof.

Without being so limited, whitening/pigmentation agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include arbutin, azealeic acid, vitamin C and its derivatives (ascorbyl palmitate, magnesium ascorbyl phosphate, sodium ascorbyl phosphate), hydroquinone, N-acetyl-4-S-cysteanimylphenol, kojic acid, melanostat (melanostatine), tretinoin, retinoic acid and its derivatives (retinal, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), ruminex occidentalis extract, licorice, mulberry, arctostaphylos uva-ursi (bearberry), tyrosinase inhibitors, melanosome-transfer inhibitors and melanin scavengers. It also includes any combination thereof.

### Kits and Packages for Extracts Compositions of the Present Invention

The extract or compositions of the present invention may be packaged in any suitable manner, including but not limited to, a jar, a bottle, a tube, a stick, a roller-ball applicator, an aerosol spray device, etc., in the conventional manner. The extract or compositions of the present invention could be packaged as a kit of two or more separate compartments, including one containing the active ingredients and a second containing a topically/dermatologically-acceptable vehicle, which may be mixed together at some fixed time point prior to application. For example, the active ingredients, in the form of a cream, a powder, a tablet, a capsule or a liquid, may be contained in sealed, single-use packets, which may be opened and mixed with the topically-acceptable vehicle, which may also be stored in pre-measured form in sealed, single-use packets. Alternatively, the active ingredients and the topically-acceptable vehicle may be provided in larger quantities from which the needed amount could be withdrawn using various measuring devices, such as a measuring spoon or cup for solids, or a calibrated vial or dropper for liquids. The extract or compositions of the present invention may be spread onto a substrate and then subsequently packaged. Suitable substrates include dressings, including film dressings, and bandages. In an embodiment, the kit or package may comprise instructions for use/application, e.g., instructions for preventing or reducing a skin condition or disorder such as a skin aging sign or scar or stretchmark.

### Subjects

The term individual, subject or patient according to this invention is intended to mean a vertebrate, preferably a mammal, more preferably a human. Mammals include, but are not limited to, farm animals, sport animals, rodents, primates, and pets.

### Prevention of Reduction of Skin Conditions or Disorders

In another aspect, the present invention provides a composition and its use (e.g., cosmetic, nutraceutical or therapeutic/pharmaceutical use) for preventing or reducing skin condition or disorder in a subject such as a skin aging sign.

In another aspect, the present invention provides a method for preventing or reducing skin condition or disorder in a subject such as a skin aging sign, comprising administering a composition comprising an effective amount of an extract on the subject's skin.

In another aspect, the present invention provides a composition and its use (e.g., cosmetic, nutraceutical or therapeutic use) for preventing or reducing a skin aging sign. Without being so limited, the term “skin condition or disorder” includes skin aging signs (including acne scars), other scars such as, but not limited to surgical scars, and burn scars, and stretchmark wounds such as stretchmarks due to pregnancies or other significant weight variations.

Without being so limited, as used herein, the terms “skin aging sign” refers to wrinkles, fine lines, scars such as acne scars, stretchmark, loss of skin firmness and elasticity, loss of texture, dehydration, weakening of skin defense mechanism, inflammation, sun damage (particularly UV radiation-induced oxidative stress), redness, telangiectasia, skin sagging, excess sebum, enlarged pores, dark circles, loss of skin firmness, brown spot, age spots, hyper pigmented skin, increased skin thickness, blemishes, loss of skin elasticity and collagen content, dry skin, lentigines, melasmas, dull skin, bags under eyes, lack of sebum, loss of skin comfort and skin devitalization (reduced metabolic activity), or any combination thereof.

As used herein, the terms “reducing” in the expression “reducing skin aging sign” or “reducing skin condition or disorder” is meant to refer to a reduction of a pre-existing aging skin sign, or other skin condition or disorder, respectively. It encompasses complete or partial correction/treatment of the aging sign or other skin condition or disorder, respectively. As used herein, the term “preventing” in the expression “preventing skin aging sign” or “preventing skin condition or disorder” is meant to refer to a delay in the initiation of, or a complete or partial prevention of a skin aging sign, or other skin condition or disorder, respectively.

In an embodiment, the above-mentioned treatment (a) modulates the expression of a gene involved in cellular senescence; (b) modulates the expression of one or more gene(s) involved in ECM (extra cellular matrix) remodeling; (c) modulates the expression of one or more gene(s) involved in apoptosis; (d) modulates the expression of one or more gene(s) involved in anti-radical defences; (e) modulates the expression of one or more gene(s) involved in anti-radical differentiation (f); improves collagen fibres thickness; (g) increases collagen tensing effect; (h) increases the expression of collagen I; (i) increases cellular energy; (j) increases cellular differentiation; (k) increases transglutaminase K activity; (l) reduces crow's feet depth; (m) reduces mouth wrinkles; (n) increases skin firmness; (o) increases skin moisture; (p) improves skin texture; (q) improves skin microrelief; (r) promotes skin repair functions; (s) increases the expression of one or more gene(s) involved in telomerase activity; and/or (t) increases telomerase activity.

In another embodiment, the composition of the present invention (a) modulates the expression of one or more gene(s) involved in ECM optimization properties, (b) modulates the expression of one or more gene(s) involved in apoptosis, (c) modulates the expression of one or more gene(s) involved in anti-radical defenses (d) increases collagen tensing effect, (e) increases cellular energy and/or (f) increases cellular differentiation.

In a further embodiment, the composition of the present invention a) increases the expression of at least one of BCL2-like 1; Colony stimulating factor 2, Cyclin F, Dermapontin, Fibromodulin, Heat shock 70 kDa protein 4, Insulin-like growth factor binding protein 5, Intercellular adhesion molecule 1, interleukin 11, Interleukin 8, Matrix metalloprotease 14 (MMP14), Neuregulin 1, Neurofibromin 1, osteopontin, cornifin, mitochondrial Superoxide Dismutase 2, Gamma synuclein, Telomerase RNA component, Telomerase reverse transcriptase, Thrombospondin 1, Thromboxane A2 receptor, Transcription factor AP-2 beta, Tumor necrosis factor receptor superfamily member 1B and Tumor protein p53; and/or b) decreases the expression of Keratin 10 and/or Keratin 19.

In an embodiment, the extract modulates apoptosis, increases anti-radical defenses, increases anti-radical differentiation and/or improves the extracellular matrix of skin cells. In a particular embodiment, the modulation of apoptosis involves a reduction in the expression of at least one of BCL2, survivin and TRAIL. In an embodiment, the increase in anti-radical defenses involves an increase in the expression of at least one of glutathion peroxidase, glutathion reductase, heme oxygenase and catalase. In an embodiment, the increase in anti-radical differentiation involves an increase in the expression of Heat shock protein 27 (HSP27). In another particular embodiment, the improvement on the extracellular matrix of skin cells involves an increase in the expression of at least one of collagen, decorin, biglycan, fibrillin, tenascin, integrin β1 and plasminogen activator inhibitor and/or a reduction in the expression of at least one of MMP1 and MMP3.

### Method of Delivery

The method of delivery of the extract or compositions of the present invention may vary, but usually involves application of the extract or composition to an area of skin prone to, or affected by, a skin aging sign, e.g., any skin sign associated with, caused by, or affected by, intrinsic aging and/or extrinsic aging or another skin condition or disorder.

A cream, lotion, gel, ointment, paste or the like containing the extract or composition may be spread on the affected surface and gently rubbed in. A solution may be applied in the same way, but more typically will be applied with a dropper, swab, or the like, and carefully applied to the affected areas.

### Dosages and Application Regimen

The expressions “effective amount” and “therapeutically effective amount” as used herein with reference to the extracts and/or compositions of the present invention are intended to mean an amount sufficient to initiate a beneficial or desired clinical result, namely a cosmetic, nutraceutical, therapeutic or prophylactic response against at least one skin condition or disorder including skin condition or disorder of the subject (e.g., effectively prevents or reduces a skin aging sign such as an improvement of the condition of the skin, reducing or preventing senescence of any cell (e.g., cosmetic and/or nutraceutical effect). It typically constitutes an amount sufficient to cover the skin that is to be treated. The effective amount may vary depending on the form of the composition (e.g., gel, cream, serum, etc.), the type of skin of the subject, the nature of the tissue, the severity of the damages, the mode of administration, etc. The dosage or application regimen will depend on a number of factors that may readily be determined, such as the severity of the condition or disorder and its responsiveness to initial treatment, but will normally involve one or more applications per day on an ongoing basis. One of ordinary skill may readily determine the optimum amount of the formulation to be administered, administration methodologies and repetition rates. In general, it is contemplated that the formulations of the invention will be applied in the range of once or twice weekly up to once or twice daily. One skilled in the art can easily and without difficulty monitor the aged or damaged tissue so as to determine what will be such an effective amount.

In an embodiment, the extract of the present invention is present in an effective amount to provide a desired result on the skin (e.g., for preventing or reducing a skin condition or disorder in a subject (e.g., skin aging sign)). In an embodiment, the extract of the present invention is present in a concentration between about 0.01 g/L to about 200 g/L of the skin care composition. In an embodiment, the extract of the present invention is present at a concentration between about 0.01% and about 5% w/w of the composition (e.g., 3% w/w as in Example 6).

The present invention is illustrated in further details by the following non-limiting examples.

### Example 1

### Preparation of a Crude Extract from Saccharina Longicruris

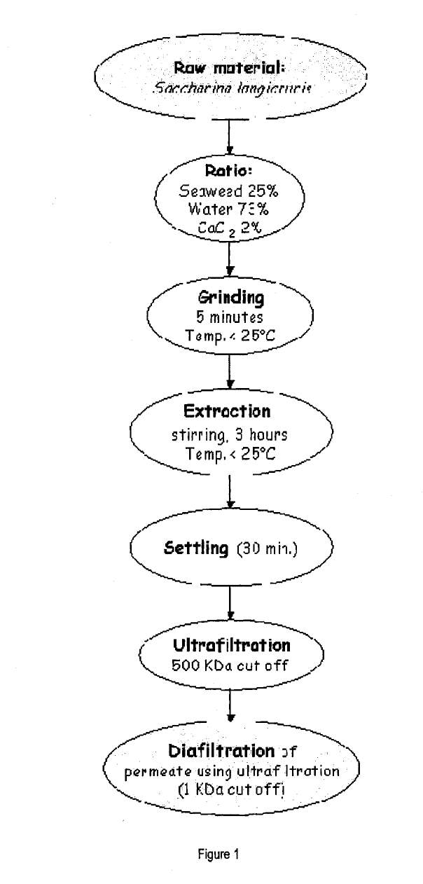

Brown seaweeds from Saccharina longicruris, were extracted according to the process outlined in FIG. 1 so as to provide a crude extract.

### Example 2

### Quantitative Genetic Profiling Studies in Senescent Fibroblasts Exposed to the Crude Extract Described in Example 1

The DNA chip technique is a unique tool to determine the effect of various substances on the modulation of the expression of genes expressing proteins of various structures and functions which are necessary to the normal functioning of the organism. For cosmetic applications, this technique allows the identification of mechanisms of action of particular interest. The cellular process of the aging dermis was studied with a senescent fibroblast cellular model.

With this method, the ability of a crude extract of Saccharina longicruris prepared with the method described in Example 1 (test compound) to regulate the expression of target genes involved in natural physiological processes of the skin (i.e., processes allowing the skin to maintain a maximum integrity at the cellular and extracellular levels) was evaluated.

### Method

Cells: Primary culture of normal human epidermal fibroblast (NHDF-Ad) at passage 8 (R8) (young or normal cells) and passage 14 (R14) (aged cells) were used. Culture medium: DMEM (Fisher 21969035); Glutamine 2 mM (Fisher 25030024); Penicillin 50 UI/ml-Streptomycin 50 μg/ml (Fisher 15070063); Fetal calf serum 10% (FCS, Fisher 10106451).—Assay medium: DMEM (Fisher 21969035); Glutamine 2 mM (Fisher 25030024); Penicillin 50 UI/ml—Streptomycin 50 μg/ml (Fisher 15070063); Fetal calf serum 1% (FCS, Fisher 10106451).

Fibroblasts (R14, aged cells/senescent or R8 young or normal fibroblast) were seeded in 12-well plates, in DMEM at 10% FCS. After 24 h of incubation, the culture medium was removed and replaced by assay medium (DMEM 1% FCS) and the cells were incubated for 24 h. The medium was removed and replaced by an assay medium containing or not (control) the test compound and the cells were incubated for 24 h at 37° C. and 5% COP. A sole dose of the crude extract of Saccharina longicruris (test compound) was tested, i.e. 0.75% w/v. This concentration had been determined by prior cytotoxicity testing.

At the end of each incubation time, the cells were washed in PBS buffer (Invitrogen 14190094), 300 μl of TriReagent™ was added to extract RNA and the cells were immediately frozen at −80° C.

DNA chips: DNA Nylon chips dedicated to the analysis of gene expression and especially suitable for screening (Product by BIOalternatives) were used. These Nylon chips (<3 cm²) were made using proprietary equipment (Piezzo Technology, Piezorray, PerkinElmer) and specific DNA targets. The analysis was made also using proprietary technology. As is well known in the art, DNA chip microarrays analysis is based on the use of mRNA, of reverse transcription and ³³P labeling.

DNA chip analysis: The quantity and quality of total cellular RNA extracted were evaluated using an Agilent 2100™ Bioanalyzer. mRNA was then isolated from the total RNA. The DNA targets were then prepared and labeled with ³³P through direct reverse transcription of the messenger RNA with (^{α33}P)-dATP and oligo dT. Labelled DNA targets were then hybridized to the DNA probes covalently attached to the chip. After several washes, the relative quantity of each hybridized target was evaluated by PhosphorImaging™.

Differential expression analysis: Gene expression analysis was performed using standard minichips dedicated to research the gene expression and especially adapted to screening purposes (produced by BIOalternatives). These Nylon chips (<3 cm²) were spotted using proprietary spotting device (non-contact spotter, piezzo technology, Piezorray, PerkinElmer) and cDNAs specific for markers of interest. The analysis was made using a proprietary technology allowing the miniaturization of the currently used formats and cost-effective analysis. It is based on the use of mRNA as a template for reverse transcription and 33P label (optimal sensitivity).

The analysis was realized by direct quantification of spot radioactivity using a Cyclone PhosphorImager™ (Packard instruments; 72 h exposition) and ImageQuant™ TL, an image analysis software (Amersham Biosciences).

### Results

In this experiment, only genes which were upregulated by more than about 200% or down-regulated by more then about 50% were considered.

Analysis of gene expression in aged fibroblasts treated with crude extract demonstrated the following (see Table 1 below):
an increase in extracellular matrix (ECM) protein genes (collagens, decorin, biglycan, fibrillin, tenascin);
a reduction in MMP-3 and MMP-1;
a increase in a gene involved in building of the cytoskeleton (Integrinβ);
an increase in gene which inhibits MMPs activation (Plasminogen activator inhibitor 2 (PAI 2));
a reduction in the expression levels of genes involved in apoptosis (BCL-2, survivin, TRAIL);
a modulation of genes associated with numerous syntheses and with cellular differentiation (Heat Shock Protein (HSP));
an increase in genes involved in anti-radical defences (catalase, glutathion peroxidase, gluthation reductases, heme oxygenases).

TABLE 1
Gene expression profiling in old/senescent fibroblasts treated
with a crude extract of Saccharina Longicruris

| | | |
|---|---|---|
| | Effect of | Effect of Crude |
| Genes | Cellular Aging | Extract |

Effect on extracellular matrix

| | | |
|---|---|---|
| MMP1, MMP3 | + | − |
| Collagen, decorin, biglycan, | − | + |
| fibrillin and tenascin | | |
| Integrin β1 | − | + |
| Plasminogen activator | − | + |
| inhibitor 2 (PAI 2) | | |

Effect on apoptosis

| | | |
|---|---|---|
| BCL-2 and survivin | | − |
| TRAIL | + | − |

Effect on anti-radical defences

| | | |
|---|---|---|
| Gluthation peroxidase and | | + |
| heme oxygenase | | |
| Gluthation reductase and | − | + |
| catalase | | |

Effect on anti-radical differentiation

| | | |
|---|---|---|
| Heat Shock Protein (HSP 27) | − | + |

− Reduction in gene expression
+ Increase in gene expression

### Conclusion

The total effects on genetic expression indicate that the crude extract has a strong potential for anti-senescence and anti-cellular aging effects.

### Example 3

### Evaluation of Fibroblast's Cellular Energy in the Presence of a Crude Extract of S. longicruris Principle

Cellular respiration takes place in mitochondria. These small organelles use oxygen to produce cellular energy which is stored in the cell in the form of ATP. This energy is then used by the cells to perform its functions. Aging cells undergo a significant decrease in the efficiency of cellular respiration leading to decrease in cellular regeneration and repair.

Cytotoxicity studies were performed on a crude extract of S. longicruris prepared using the method disclosed in Example 1 to assess its capacity to generate an increase in cellular metabolism.

### Method

Biological material: Primary culture of normal human fibroblasts R8 (NHDF-Ad) were used.

Test compound: Doses of crude extract of Saccharina longicruris 0.375, 0.75, 1.5 and 3% w/v.

Assessment of mitochondrial activity: Fibroblasts were grown in the presence or absence (control) of the test compounds for 24 hours. After the incubation period, cellular integrity and cellular morphology were evaluated (data not shown). An additional test was performed to evaluate cellular viability using the MTT standard method.

### Cellular Respiration

### Results

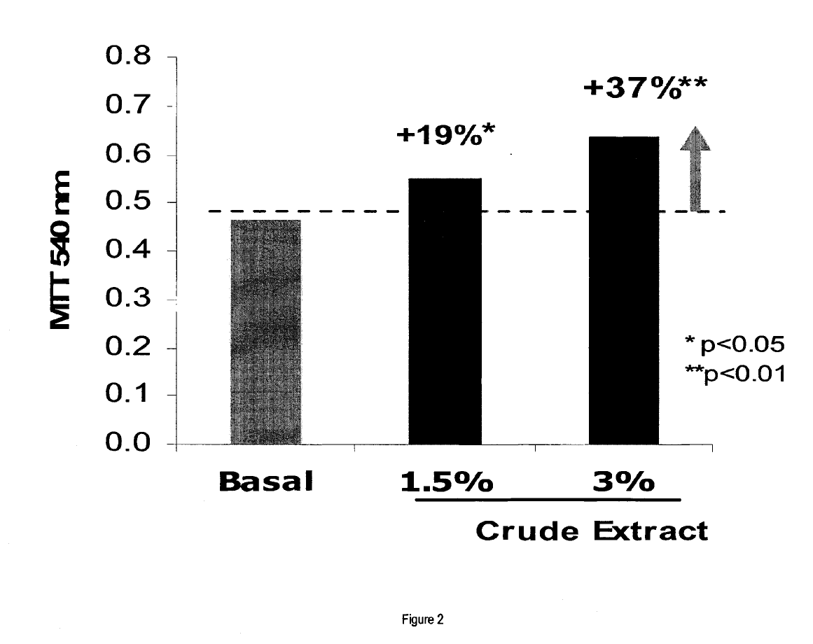

The results presented in FIG. 2 show the effect of the crude extract on cellular metabolism of fibroblasts grown in monolayers. The crude extract has a dose/response effect on mitochondrial activity with a significant increase of 19% and 37% on cellular respiration in fibroblasts treated with the crude extract at doses of 1.5 and 3%, respectively.

Cells exposed to the crude extract show a significant increase in mitochondrial activity. This increase correlates with a profile of young cells, able to function adequately and to regenerate.

### Example 4

### Determination of the Effect of a Crude Extract of Saccharina longicruris on Lattice Contraction

### Principle

One of the most important factors of cutaneous aging involves loss of skin elasticity, the visible sign of which is the appearance of wrinkles and fine lines. Cutaneous aging is due to loss of extracellular matrix (ECM) integrity combined to a reduction in skin structure organization. This results in a change in skin texture and appearance.

Collagen gels containing fibroblasts contract due to the traction of these cells on collagen fibres without any outside intervention. Modeling of this contraction is made through cellular culture techniques and it is possible by associating fibroblasts and collagen to rebuild a live tissue generally known as equivalent dermis (ED). This contraction phenomenon is measured experimentally in terms of a reduction of the diameter of a collagen gel disk. (Fernandes et al. 1999). Interconnected in a collagen matrix (lattices), aging cells exposed to the crude extract can therefore be tested for their ability to induce contraction of a collagen network as compared to that performed in younger cells.

The ability to restore collagen contraction in lattices in which contraction had been diminished by senescence has been tested with the crude extract prepared as described in Example 1.

### Protocol

Biological material: Collagen (1.3 mg/ml rat-tail collagen, Institut J. Boy) and primary cultures of passage 8 (young cells), or passage 14 (aged cells), fibroblasts (NHDF) were used. The crude extract of S. longicruris was tested at a dose of 0.08% (test compound).

Evaluation of lattice contraction: The lattices were prepared by mixing collagen and a cellular suspension of NHDF. Two types of lattice were prepared: a “young” matrix with passage 8 (R8) NHDF's and a second “aged” matrix with passage 14 (R14) NHDF's.

After lattice polymerization, the culture medium was replaced by fresh medium with and without the test compound or the TGFβ reference product. Lattices were then incubated for a period of 4 days. The surface of each lattice was evaluated at day 1 (D1) and day 4 (D4) by image analysis.

### Results

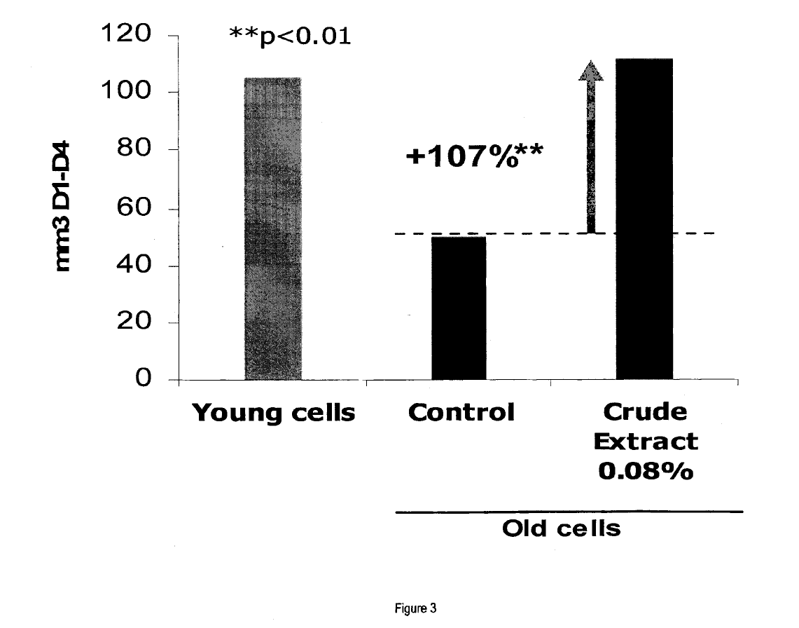

Results shown in Table 2 below and FIG. 3 present the ability of the crude extract to induce a contraction of the aged cell lattices. Contraction is apparently restored to the level of contraction of young cells after 4 days of treatment with the crude extract. Indeed, on day 4, a contraction percentage identical to control R8 (young cells) was observed, i.e. two times more than in the R14 control.

TABLE 2
Ability of a crude extract of S. Longicruris to
induce cell contraction

| | | |
|---|---|---|
| | Lattice Surface | |
| Lattice | mm² | % Contraction |

| Characteristics | Day 1 | Day 4 | D4/D1 |
|---|---|---|---|
| Young cells (R8) | 404 | 299 | 26.0 |
| Aged cells (R14) | 368 | 318 | 13.6 |
| Aged cells + | 423 | 311 | 26.5 |
| 0.08% | | | |
| crude extract | | | |

Aged cells treated with the crude extract recovered their ability to induce contraction of the collagen network to the level of that of young cells.

### Example 5

### Determination of the Effect of a Crude Extract of S. Longicruris on Cellular Differentiation

### Principle

Keratinocyte transglutaminase (TGK) enzymatic activity is a cellular hallmark of keratinocytes differentiation. An increase in transglutaminase activity in keratinocytes cultures indicates that the cells are differentiating. This cellular state is part of the normal regeneration process of the skin, which allows its reinforcement thereby limiting water evaporation, pollution effects as well as protecting it from cold weather and UV rays.

### Protocol

Biological Material: Keratinocyte culture (NHEK cells). The crude extract of S. longicruris prepared as described in Example 1 was tested at the following concentrations: 0.75%, 0.25% and 0.08% (test compounds). The positive control (CaCl₂) was tested at 1.5 mM.

Method: NHEK cells were grown up to 80% confluence. The test compounds and control were directly added to the culture medium and incubated for 96 hours. TGK was extracted from cell membranes and its activity was measured by covalently adding ³H-putrescin (Amersham TRK 144; 1.04 TBq/mmol, 28 Ci/mmol; 2 μCi/ml final) to casein (protein acceptor, 2 mg/ml final). Radioactivity was then measured in the precipitate, by liquid scintillation. In parallel, the total quantity of proteins in each sample was determined using the Lowry method.

### Results

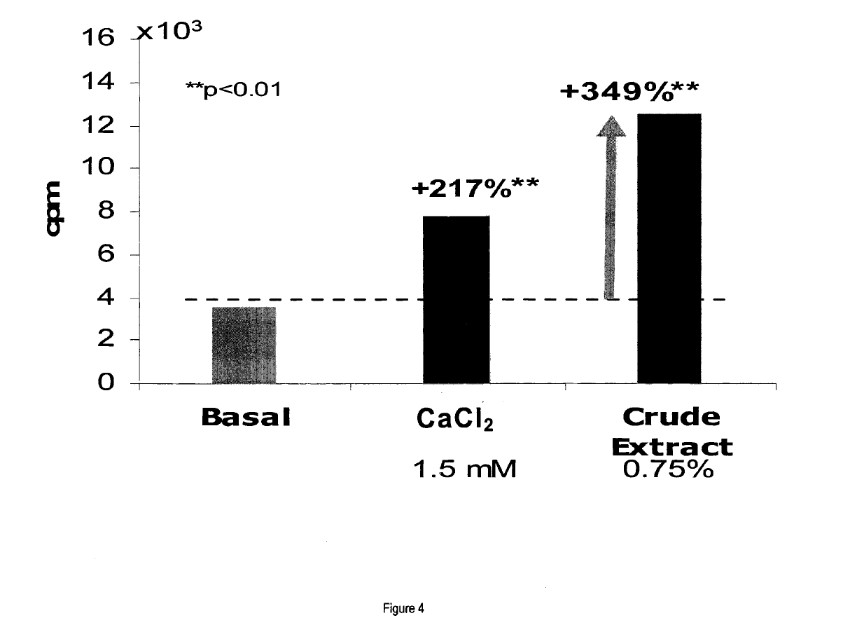

The results presented in Table 3 below and FIG. 4 show that the crude extract increased TGK activity by more than 349% as compared to basal TGK activity.

TABLE 3
Differentiation level of keratinocytes (TGK) following
treatment with a crude extract of S. longicruris

| | TGK Activity | |
|---|---|---|
| Extract | (Control %) | p |
| Control (+): | 217 | p < 0.01 |
| CaCl₂ 1.5 mM | | |
| Crude Extract | 349 | p < 0.01 |
| 0.75% | | |

### Conclusion

The ability of the crude extract to stimulate TGK activity indicated the crude extract's pro-differentiation effects.

### Example 6

### Cosmetic Performance of a Crude Extract of S. Longicruris as an Anti-Aging Care Product Assessed Through Consumer Testing

### Principle

The purpose of Consumer Testing is to investigate the product performance with target consumers in real-life conditions. The purpose of these consumer tests is to assess the cosmetic performance at both preventive and curative levels in normal use conditions. In such a context, the product safety is also confirmed in standard use conditions.

Test: 120 female volunteers aged between 40 and 75 used the product at home for a period of 4 weeks. Female consumers were asked to use the test product twice a day for 4 weeks without changing their cosmetic routine. They were also asked to refrain from using other cosmetic cares during the test. Two auto-tests were filled in on Day 7 and Day 28 at the end of the study. Consumers returned the questionnaires by mail. Volunteers potentially eligible for the test were selected out of an Iris computer data bank (specifically built for anti-aging applications).

Tested Product: Formulation described in Table 4 below containing 3% w/w of the crude extract, the crude extract being prepared as described in Example. The pH of the composition was 5.3 with a viscosity of 52000 cps at 25° C. 3 days after manufacture. The composition was stable after 4 days at 45° C.

TABLE 4
Formulation used in tests with volunteers

| Ingredient | Supplier | % w/w |
|---|---|---|
| Demineralized Water | | q.s. à 100% |
| Ultrez-21 Carbopol ™ | Noveon | 0.30 |
| (Acrylates/C10-30 Alkyl Acrylate | | |
| Crosspolymer) | | |
| Butylene Glycol | Brenntag | 5.00 |
| Phenonip ™ | Nipa | 0.80 |
| (Phenoxyethanol, Methylparaben, | | |
| Ethylparaben, Butylparaben, | | |
| Propylparaben, Isobutylparaben) | | |
| Cutina MD ™ | Cognis | 4.00 |
| (Glyceryl Stearate) | | |
| Cetyl Alcohol | Croda | 2.00 |
| Cetiol CC ™ | Cognis | 3.50 |
| (Dicaprylyl Carbonate) | | |
| USP Light Mineral Oil | Penreco | 5.00 |
| (Paraffinum Liquidum) | | |
| Tween 60 ™ | Uniquema | 0.75 |
| (Polysorbate 60) | | |
| SF-96-100 | G.E. | 0.70 |
| (Dimethicone) | | |
| Perfume No. 1457“Ozone marin”/Sea Ozone | Fleurarôme | 0.03 |
| (Perfume) | | |
| Triethanolamine 99 | Dow | p.s. |
| | Chemical | |
| Saccharina longicruris crude Extract | Innovactiv | 3.00 |

### Results

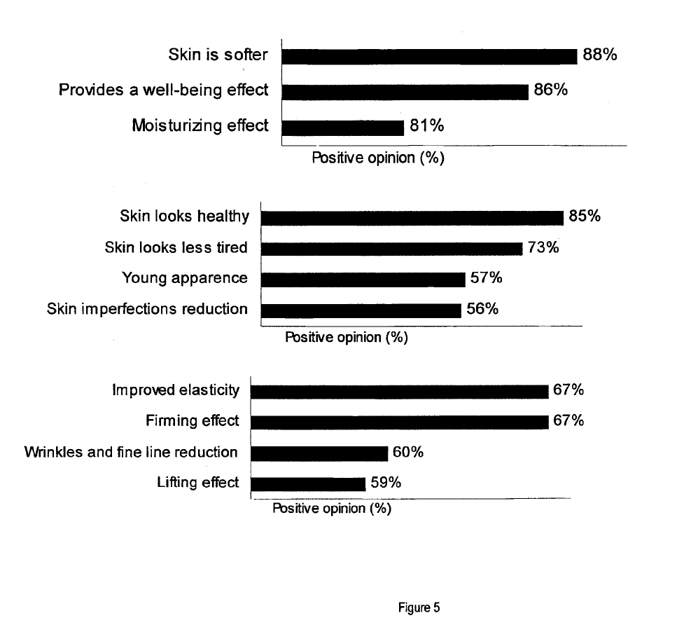

The data from the questionnaires filled in at the end of the test highlighted the following points concerning this product, especially when comparing frequencies observed for the test product with corresponding frequencies for products of the same class registered in the Iris database (see FIG. 5).

The test product was said to:
leave the skin smooth (significantly better than past history of yes/no questions)
have a hydrating effect (significantly better than past history of yes/no and open questions)
have a pleasant texture (significantly better than past history of yes/no questions)
give the skin a feeling of well-being (significantly better than past history of yes/no questions)
does not feel greasy (significantly better than past history of yes/no questions)
penetrate quickly
leave the skin soft (also observed in past history of yes/no questions)
leave the skin more comfortable all day long (significantly better at D28 than D7)
give an even-toned complexion (significantly better at D28 than D7)

Furthermore, the anti-aging care product was safe under the conditions tested.

A moisturizing effect leaving the skin smoother and softer was observed. The test product made the skin firmer and younger-looking by reducing fine lines and imperfections.

### Example 7

### Determination of the Mutagenic Activity of a Crude Extract of S. longicruris

### Principle

The Ames test is a biological assay to assess the mutagenic potential of chemical compounds. A positive test indicates that the chemical might act as a carcinogen. As cancer is often linked to DNA damage, the test also serves as a quick assay to estimate the carcinogenic potential of a compound. The assessment of the genotoxic potential of a chemical substance has become an essential component of the approval process required by regulatory authorities in the cosmeceutical Industry.

Mutagenicity was measured using the AMES-II™ kit with S9 liver fraction (Aniara) according to the manufacturer instructions. Point Mutations were made in the histidine (His) operon in Salmonella typhimurium, rendering the bacteria incapable of producing histidine. These mutations are positioned at strategic points within the His gene, resulting in his-organisms that cannot grow unless histidine is supplied. When a mutagenic event occurs, base substitutions or frameshifts within the His gene may cause a reversion to histidine prototrophy. These reverted bacteria will then grow in histidine-deficient media. The crude extract's mutagenic potential was assessed by exposing these his-organisms (TAMix™ (equimolar amount of TA7001-TA7006) and TA98 strains) to varying concentrations of extract and selecting for the reversion event. Medium lacking histidine is used for this selection which allows only those cells that have undergone the reversion to histidine prototrophy to survive and grow. This kit also contains TA98 for the detection of frameshift mutations.

Approximately 10⁷ his-bacteria were exposed to 6 concentrations (30%, 10%, 3.3%, 1.1%, 0.37 and 0.12% w/v) of the crude extract of S. longicruris prepared as described in Example 1, as well as a positive and a negative control, for 90 minutes in medium containing sufficient histidine to support approximately two cell divisions. After 90 minutes, the exposure cultures were diluted in pH indicator medium lacking histidine, and aliquoted into 48 wells of a 384-well plate. Within two days, cells that had undergone the reversion to Histidine prototrophy were grown into colonies. Metabolism by the bacterial colony reduces the pH of the medium, changing the color of that well. The number of wells containing revertant colonies were counted for each dose and compared to a zero dose (solvent) control. Each dose was done in triplicate to allow for statistical analysis of the data. An increase in the number of revertant colonies upon exposure to test chemical relative to the zerodose controls indicated that the chemical was mutagenic in the Ames II assay.

### Results

The S. longicruris crude extract is non-mutagenic for bacterial strains under the tested conditions. A series of 6 dilutions 1:3 were tested (30%, 10%, 3.3%, 1.1%, 0.37 and 0.12% w/v) in the presence and in the absence of a final concentration of 30% of the enzymatic S9 fraction. Only the positive controls gave positive results.

### Example 8

### Primary Ocular Tolerance of a Crude Extract of S. longicruris

The HET-CAM Test (Hen's Egg Test—Chorioallantoic Membrane) is a qualitative method for assessing product irritation potential and is used as an alternative to the Draize test (tolerability test on animals).

Ten days old fertilized eggs divided in 6 test groups were used: 3 concentrations of the crude extract prepared as described in Example 1 (10, 20, 30% w/v), 2 positive controls and 1 negative control. By using whole tissue containing arteries, veins and capillaries it is possible to mimic an eye inflammation in the presence of a potentially irritating substance.

Eggs were incubated upon receipt. After 10 days of development, eggs were taken out of the incubator and the viability of the embryo verified. A rectangular opening was made in the shell in order to free the membrane. The free part was humidified using 2-3 mL of 0.9% saline water and the eggs were put back into the incubator. The safety of the extract was then analyzed. Once the extract was added, the eggs were constantly observed for 5 minutes to identify any signs of lysis, haemorrhage or coagulation.

Results showed that the extract is non-irritating at concentrations of up to 30% w/v.

### Example 9

### Determination of Skin Tolerance and Skin Sensitization in Healthy Volunteers

The Human Repeat Insult Patch Test (HRIPT) allows to determine skin tolerability as well as potential skin sensitization (allergy or contact dermatitis) for a product intended for tropical applications. The crude extract prepared as described in Example 1 (33% w/v) was applied 10 times in a 3-week period in blotter form on the skin of 100 healthy volunteers. Contact duration between applications was 48 hours. After a rest period of 10 to 14 days, volunteers were once more exposed to the extract for a period of 48 hours to validate the absence of allergic reaction. Tests were supervised by a dermatologist.

### Result and Conclusion

The extract was tested at a dilution rate of 1:3 (33% v/v). Test results indicate that under the testing conditions, the product does not induce a skin irritation or intolerance reaction nor has a skin sensitization reaction been observed. In conclusion, the extract is not considered as an irritant.

### Example 10

### Stability of a Solution of 30% w/w of Crude Extract in Glycerol

A solution comprising 60 ml of S. longicruris crude extract prepared as described in Example 1 and 140 ml of glycerol was prepared and its stability was assessed. Solutions were put into hermetically closed glass vials.

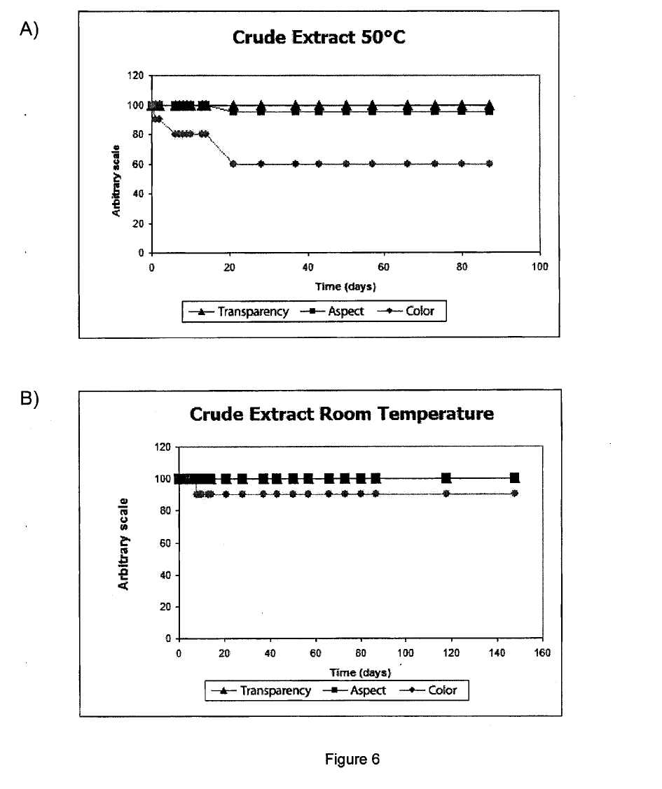

Stability at room temperature: Excellent stability in terms of color as much as aspect and clarity after 150 days (see FIG. 6A). The solution is still under study and will remain so for up to three years. The solution that has been put through a freezing cycle was as stable as that kept at room temperature after 150 days.

Stability at 50° C.: Excellent stability in terms of texture as much as clarity. The color of the solution had darkened significantly at the beginning of the study; reaching a light brown at Day 20. Then, the color was stabilized, resulting in an acceptable appearance (see FIG. 6B).

### Example 11

### Preparation of a Purified Laminaran Extract from S. longicruris

The Saccharina longicruris seaweeds were milled in a Desintegrator (Rietz Manufacturing Co., Santa Rosa, USA) fitted with perforated plates of 4.65 mm, vacuum sealed and kept at −30° C. until use.

Crude laminaran was extracted from the milled seaweeds (Nishino et al. 1989). Briefly, 150 g of seaweeds were mixed with HPLC grade water and 1% (w/v) of CaCl₂. The mixture was stirred at 85° C. during 4 hours and then, centrifuged (16 887 g, 20 minutes). The supernatant was vacuum filtrated with Whatman #4 filters. The filtrate was mixed with 1 volume of NaCl 2% and 2 volume of ethanol 95% and then, stirred during 1 hour at room temperature and stored at −20° C. during 48 hours. The mixture was centrifuged (16 887 g, 10 minutes) to isolate the pellet (native fucoidan) and the supernatant (crude laminaran fraction containing a mix of laminaran, fucoidan and polyphenol) separately. The procedure was performed a number of time to produce many fractions. The ethanol was evaporated from the supernatant and then, all fractions were dialysed with 1 kDa membranes, freeze-dried and kept at 4° C. until use.

Crude laminaran fractions were resuspended in water at a concentration of 0.1% v/v. Laminaran was purified by ultrafiltration with a 50 kDa Prep/Scale-TFF membrane (Millipore, Canada) to remove fucoidan from the mixture. The filtrates were freeze dried. The filtrates were resuspended in water and added on a low pressure column with Amberlite resin (Sigma, Canada) to remove all traces of polyphenols. Purified laminaran fraction was eluted with 5 column volume of water and polyphenol was isolated with 5 column volume of ethanol 95%. Laminaran solution was freeze-dried to obtain a powder.

### Example 12

### Structural Characterisation of the Purified Laminaran Extract

### Monosaccharides Analysis

After acidic methanolysis of the laminaran and subsequent GC analysis, neutral sugars were identified as trimethylsilyl derivatives. Methanolysis was performed in 2 M MeOH—HCl (Sigma, Canada) at 100° C. for 2 h and the methyl glycosides were converted to the corresponding per-O-trimethylsilylated derivatives (Kemerling et al. 1975, Montreuil et al. 1986). The derivatives were separated and quantified by GC on a HP 5890A system equipped with a FID detector and a CP-Sil-5CB fused silica column (60 m×0.25 mm, Chrompack, Varian) in a split/splitless mode using helium at a flow of 1.5 mL/min as the gas carrier. The oven temperature was programmed at 50° C. for 1 min, 20° C./min to 120° C., and then 2° C./min to 240° C. The injector and detector temperatures were maintained respectively at 290° C. and 300° C. The monosaccharides were identified according to their retention times and quantified using an internal standard method involving myo-inositol. All analyses were made in triplicates.

### Methylation Analysis

Methylation analysis was performed using a modification of three methods (Hakomori 1964, Hellerqvist 1990, Waeghe et al. 1983). First, laminaran (2 mg) was treated with Dowex™ cation exchange resin (Sigma, Canada) and freeze-dried. Then, laminaran hydroxyl groups were methylated with methyl iodide in dimethyl sulfoxide (Sigma, Canada) and with lithium 1-butanide (Sigma, Canada) as anion. Methylated laminarans samples were purified with Sep-Pak™ C18 (Waters, Canada) eluted with acetonitrile and dried under nitrogen flow. The purified methylated laminaran was hydrolysed with 2M trifluoroacetic acid for 2 h at 100° C. and reduced with NaBD₄. Then, derivatives were acetylated with acetic anhydride (Sigma, Canada) and pyridine (Sigma, Canada). Samples were diluted with methylene chloride (Sigma, Canada) prior to analysis. Samples were analysed by gas chromatography-mass spectrometry (GC/MS). A CP-Sil-5CB fused silica column (60 m×0.25 mm, Chrompack, Varian) using helium as carrier at a flow rate of 1.2 mL/min. The oven temperature was programmed at 50° C. for 1 min, 20° C./min to 120° C., and then 2° C./min to 240° C. Injector and detector temperatures were maintained respectively at 290° C. and 300° C. The monosaccharides were identified according to their retention times and mass fragments. Quantification was realized using an internal standard method involving myo-inositol. Ionization was carried out in electron impact (EI, 70 eV).

### Electrospray Ionisation Mass Spectrometry

Sample preparation and data analyses were performed using a modification of the Read method (Read et al. 1996). Briefly, samples (2 mg) were pre-treated with Dowex™ cation exchange resin (Sigma, Canada) and methylated. Then, samples were purified with Sep-Pak™ C18 (Waters, Canada) eluted with acetonitrile. Samples were prepared by adding 50% of the acetonitrile mix and 50% solution containing 0.01% sodium acetate in 50:50 v/v water:MeOH.

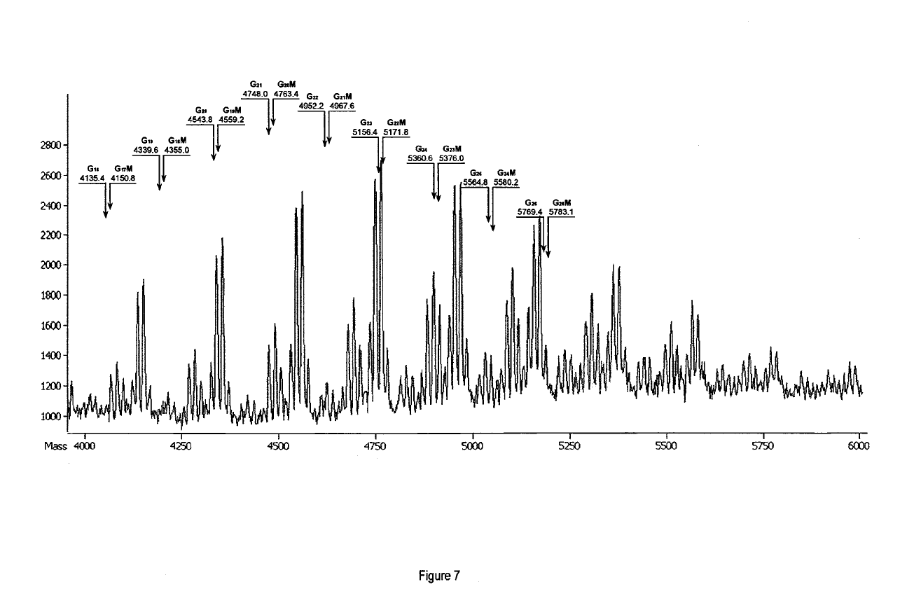

Analyses were performed on a Leco™ High-Throughput Unique Time of flight (TOF) mass spectrometer (MS). The instrument was equipped with a syringe direct injector (Harvard) at a flow rate of 50 uL/min. The flow was interfaced with an high flow atmospheric pressure ionization electrospray (ES) chamber. Conditions for the analysis in the positive ion mode included an electrospray voltage of 2 kV, a nebulizer pressure of 250 kPa and a desolvation temperature of 210° C. Data were collected on ChromaTOF™ (Leco) over a mass range of m/z 50-6000 at 2.08 spectra per second. The instrument was calibrated using an ES tuning Mix™ (Agilent technologies). Since the highest calibration point within the mix was 2721.89, the calibration was slightly adjusted to give G23 at the expected point. Then, the other masses were determined from this point. In order to improve the sensibility, around 1250 scans were acquired and averaged. Results are presented in FIG. 7. Each peak provides information the sugars' respective locations in the laminaran molecule structure.

### HPSEC

Polysaccharides weight average, Mw, was determined by HPSEC (High Performance Size Exclusion Chromatography). The HPSEC system consisted of a Beckman™ 126 HPLC pump (Beckman Coulter Canada, Inc., Mississauga, ON, Canada), an injection loop of 100 μl, an analogue Beckman™ interface module Gold™ 406 (Beckman Coulter Canada, Inc., Mississauga, ON, Canada) and a Hitachi™ L-7490 refractometer (sensibility ½) (Hitachi High-Technologies Canada, Inc., Rexdale, ON, Canada). The molecular weight values were analysed with Gold™ software version V810 with molecular weight function. Molecular weight calibration curves was realized with 5 polyethylene glycol (PEG) standards from 985 to 12 400 Da (Phenomenex, inc., Torrance, Calif., USA).

Two columns were used in line: TSK-guard column PWXL (6 mm×40 mm) and a TSK-G3000 PWXL (7.5 mm×300 mm) (Tosoh Bioscience, Montgomeryville, USA). Molecular weight separation range for polysaccharides was established from 1 to 60 kDa by the supplier. The mobile phase consisted of 0.1M NaCl solution realized with HPLC grade water filtrated on 0.22 μm filters. The flow rate was 0.8 ml/min and analyses were performed at room temperature. Samples were dissolved in 0.1M NaCl solution to reach a concentration of 1 mg per ml and filtrated on 0.45 μm filters to eliminate dust particles.

### Results

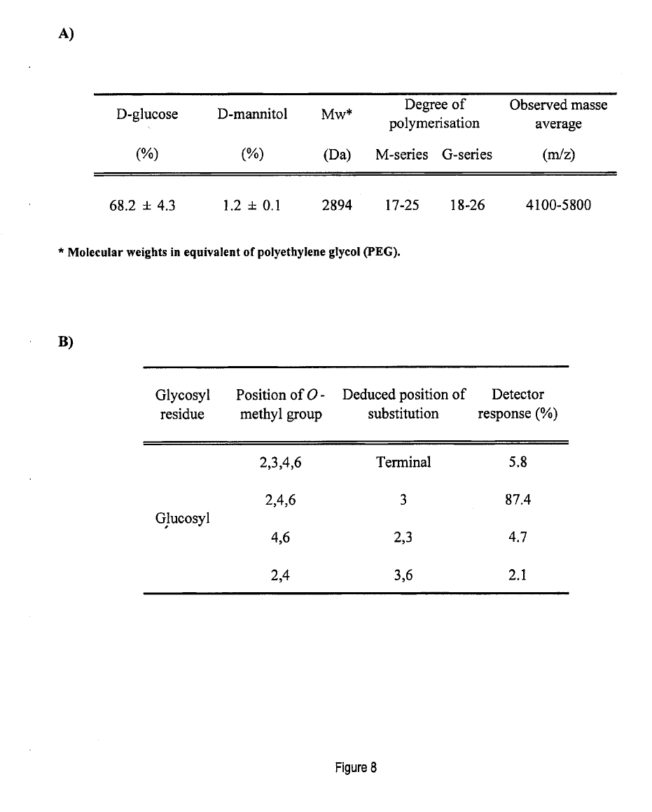

The purified laminaran extract prepared as described in Example 11 comprises 68.2% of D-glucose and 1.2% of D-mannitol. Linkage analysis showed the presence of a glucosyl linked at position (O-3) on the backbone in a proportion of 87.4%, the remaining 12.6% glucosyl were linked as position O-6. The presence of D-mannitol was detected at a concentration lower than 0.5% (data not shown). Branching residues through O-6 and O-2 in a proportion of 2.1% and 4.7% respectively were determined (see FIG. 8). The amount of branched residues is equivalent (5.8%) to the terminal one, indicating that there is not more than one branching per chain. The laminaran has a measured molecular weight determined by HPSEC of 2894 Da expressed in equivalents of polyethylene glycol (PEG) standards or from 4100 to 5800 m/z by electrospray ionization mass spectrometry (ESIMS).

Two types of laminaran have been described in the literature, one type with chains that are terminated by D-mannitol residues (M-series) and the other type with chains terminated by D-glucose residues (G-series) (Nelson and Lewis 1974). A degree of polymerization of 17-25 (M-series) and 18-26 (G-series) was found with a ratio of M:G-series of 1:1 (FIG. 8A).

### Example 13

### Gene Expression Profiling in Human Dermal Fibroblasts Following Treatment with a Purified Laminaran Extract

The purified laminaran extract prepared as described in Example 11 was tested for gene expression profiling using DNA microarray.

### Cytotoxicity Assessment of Laminaran Extract

Human dermal normal fibroblasts (HDFs, BJ cells ATCC 2522) were classically grown in DMEM (Dulbecco's Modified Eagle's Medium) (Invitrogen, UK) with 10% of fetal calf serum (Invitrogen, UK) and 100 μg/mL of penicillin/streptomycin. Cells were maintained in a humidified incubator at 37° C. with 5% of CO₂ atmosphere. Cells were kept in culture under exponential growth conditions and were harvested with trypsin/EDTA solution. BJ cells were plated in 24-wells plates at 30 000 cells/well.

Cell viability was tested by applying the purified laminaran extract at a concentration of 0.004 to 2.5 mg/mL in cultured cells for 24 h to determine the optimal concentration for the gene expression study. A solution of 2.5 mg/mL of MTT (3-(4,5-dimethylthiazol-2-yl)2,5-diphenyl tetrazolium bromide) (Sigma-Aldrich, Germany) was applied and incubated for 2 h at 37° C. in a humidified incubator with 5% CO₂. Medium was removed and lysis solution was added. After 2 h at 37° C., 200 μL of lysis solution was transferred in 96-wells plates and read at 570 nm in a microplate reader. Cytotoxicity was expressed in percent relative to untreated cells control as a decrease of mitochondrial dehydrogenase measured by formazan production from MTT. Cell viability was assessed in triplicates for each harvest period. High cell survival was observed at 2.5 mg/mL which was the concentration used for the gene expression experiment.

### Cell Culture

For this experiment, 500 000 HDFs cells were plated in 75 cm² flasks. At 80% confluence, the cells were treated with the purified laminaran at a concentration of 2.5 mg/mL for 24 hours. Three cell cultures were tested for each laminaran extract.

### Extraction of Total RNA

Total RNA extraction was performed with RNAgents™ Total RNA Isolation System (Promega). The culture medium was decanted from each flask and the cells were washed 2 times with ice-cold sterile 1×PBS. Cells were lysed with 300 μL of chilled denaturing solution and scrapped. The lysed cells were transferred in an RNAse-free tube and homogenized. For the RNA extraction, 30 μL of 2M sodium acetate was added in the lysate and mixed thoroughly by inverting the tube 4-5 times. 300 μL of phenol:chloroform:isoamyl alcohol was added to the tube, mixed carefully by inversion 3-5 times and then shook vigorously for 10 seconds. The tubes were chilled on ice during 15 minutes. This mixture was centrifuged at 10 000 g during 20 minutes at 4° C. The top aqueous phase, that contained the RNA, was removed carefully and transferred to a new RNAse-free tube. RNA precipitation was realized by adding an equal volume of isopropanol to the aqueous phase and incubated for 30 minutes at −20° C. The RNA pellet was isolated by centrifugation at 10 000 g for 10 minutes at 4° C. The pellets were washed by adding 1 mL of ice-cold 75% ethanol and centrifuged at 10 000 g for 10 minutes at 4° C. When the pellets were dried, they were resuspended in an appropriate volume of nuclease-free water and stored at −80° C.

### RNA Integrity and Concentration Verification

The integrity and quality of RNA template were checked by spectrophotometric measurement and by Agilent™ Bioanalyzer 2100 analysis. The concentration was determined by using an aliquot of the isolated RNA diluted in TE (10 mM Tris-HCl (pH 8), 1 mM EDTA). Its concentration was determined by Nanodrop™ (Isogen ND-1000) against TE as blank solution. The integrity of total RNA was assessed by visualization of intact ribosomal RNA bands. Total RNA from higher eukaryotes should have a ribosomal band size of 1.9 kb for the 18S-RNA and 4.7 kb for 28S-RNA.

Reverse Transcription and Incorporation of a Biotin-dNTP Mixture During cDNA Synthesis

For the cDNA synthesis with biotin labelling, a reaction mix (1) was prepared with 10 μg of total RNA, Internal Standard mix-Silverquant™, and Oligo(dT)12-18 Primer (0.5 μg/mL) for a final volume of 10 μL. The reaction mix was incubated at 70° C. for 10 minutes and chilled on ice for 5 minutes. A second reaction mix (2) was prepared with 5× First-Strand™ buffer, DTT (0.1 M), Biotin-dNTP and RNaseOut™ (40 U/μL). 9 μL of reaction mix 2 was added to reaction mix 1 (10 μL) on ice and incubated at room temperature for 5 minutes. SuperScript™ II Reverse Transcriptase (200 U/μL) was added to the reaction two times and incubated at 42° C. for 90 minutes and then incubated at 70° C. for 15 minutes. RNase H (2 U/μl) was added to the reaction and incubated at 37° C. for 20 minutes. The reaction was ended by heating to 95° C. for 3 minutes. The samples were placed on ice and then hybridization was performed.

### Hybridization on DualChip™ Human Aging (Eppendorf, 0038 950 003)

The DualChip™ Human Aging contains 227 genes involved in stress and aging related pathways (extracellular matrix, keratinocyte differentiation, DNA damage, oxidative stress, cell signalling, cell proliferation, DNA repair, apoptosis, and inflammation) and 13 housekeeping genes for data normalization (complete gene list on the chip is available at www.eppendorf.com/int/index.php?sitemap=1&pb=f67783daf8290cf&action=document&contentid=1&docnode=32440&docnode=32441). There were control probes for six different internal standards used to determine the quality for both the labelling reaction, the hybridization, and to normalize the data. Several controls were present on the chip to check the hybridization step, the orientation of the array, and unspecific background. The method was based on a system with two arrays (a control and a test) per glass slide with three identical subarrays per array. Positive and negative controls were spotted on each subarrays in order to control the reliability of the experimental data.

The hybridization mix was prepared in the following order: Biotin-labeled cDNA, Biotin-HybControl™ RNase-free water, Silverquant™ hybridization additive, HybriBuffer™ A and HybriBuffer™ B for a final volume of 100 μL. The hybridization mix was heated for 5 minutes at 60° C. 100 μL of hybridization mix was dispensed slowly into the injection port of the hybridization frame to avoid inserting air bubbles. The surface around the injection port of the hybridization frame was carefully dried. The aluminum sealing pad was applied to the top of the hybridization frame to seal the injection port. Immediately after the hybridization frames sealing, the DualChip™ was placed in the thermoblock for slides. The thermoblock lid was closed and the DualChip™ slides were incubated overnight (12-16 hours) at 60° C. in the thermomixer with a mixing frequency of 1400 rpm.

### Silverquant™ Detection

After the hybridization, the slides were removed from the thermoblock and incubated for two minutes at room temperature. Both hybridization frames were removed and the slides were immediately immersed in the wash buffer. The slides were washed in three one-minute sessions. The wash buffer was removed and, the slides were incubated in pre-blocking buffer for 10 minutes at room temperature. The pre-blocking buffer was removed and the slides were incubated in the diluted gold-conjugated Anti-biotin antibody for 45 minutes at room temperature under gentle agitation (300 rpm). After incubation with the gold-conjugated Anti-biotin antibody, the box was emptied and the slides were washed in four one-minute sessions using wash Buffer. The slides were incubated with rinsing buffer for one minute. Correct amount of Silverquant™ solution and Silverquant™ solution A were added and slides were incubated for 5 minutes at room temperature under agitation (300 rpm). At the end of these 5 minutes, the staining solution (pink-grey solution) was removed, the slides were washed twice 1 min using distilled water at room temperature. The backside of the slides and the barcode sticker were wiped using a paper towel, then immediately the slides were centrifuged for 5 minutes at 600 rpm to dry.

### Array Scanning

After staining, the array was scanned using Eppendorf SilverQuant™ scanner and following SilverQuant™ software recommendations. The SilverQuant™ Analysis software was then used for spot finding and quantification.

### Data Quantification

Array images obtained were normalized and quantified using SilverQuant™ Analysis software, subsequent to the following steps:

1) Background subtraction. Fluorescence intensity of every DNA spot was determined by using the local background subtractions. A signal at least 2.5 fold higher then the local background was accepted. An intensity ratio between the sample and the test were determined by averaging the intensity of the triplicate DNA spot on the chip.

2) Internal Standards Normalization. For each array, the experiment was compared to control, corresponding to RNA from untreated cells. After spot detection, normalization with internal standards was performed. Internal standards were composed of external plant RNA added in the mix of the reverse-transcription reaction, allowing the evaluation of its efficacy. Ratios of internal standards were used to evaluate efficacy. The ratios should be included between 0.75 and 1.25. Outside these ranges, it can be concluded that the reverse-transcription reaction was not equally efficient in the control and the experiment, and that gene quantification would not be correct or artefactual.

3) Housekeeping Genes Normalization and calculation of expression ratios for each gene. A second step of normalization with 13 housekeeping genes was realized because internal standard does not include purity and quality of the RNA. The variance of the normalized housekeeping genes was used to provide an estimate of the expected variance. This led to a predicted confidence interval for testing the significance of the ratio obtained. Ratios were considered significantly different when they were outside the 95% confidence interval (Anova) (de Longueville et al. 2002, de Magalhaes at al. 2004). Only significant genes were presented. Also, an average value was calculated from the normalized ratios of the three replicates.

TABLE 5
Genes significantly modulated (overexpressed or underexpressed)
in fibroblasts treated with purified laminaran extract

| | | Swissprot | | |
|---|---|---|---|---|
| | | accession | Laminaran | |
| Gene name | Symbol | number | Average | SD |
| BCL2-like 1 | BCLX | Q07817 | 1.7 | 0.7 |
| Colony stimulating factor 2 | GMCSF | P04141 | 2.1 | 0.4 |
| Cyclin F | CCNF | P41002 | 2.3 | 0.7 |
| Dermatopontin | DPT | Q07507 | 2.4 | 1.2 |
| Fibromodulin | FMOD | Q06828 | 2.3 | 1.0 |
| Heat shock 70 kDa protein 4 | HSP70 | P34932 | 1.9 | 0.1 |
| Insulin-like growth factor | IGF5 | P24593 | 2.1 | 0.2 |
| binding protein 5 | | | | |
| Intercellular adhesion | ICAM-1 | P05362 | 2.1 | 0.5 |
| molecule 1 | | | | |
| Interleukin 11 | IL11 | P20809 | 2.0 | 0.1 |
| Interleukin 8 | IL8 | P10145 | 10.5 | 12.8 |
| Keratin 10 | KRT10 | P13645 | −2.9 | 1.4 |
| Keratin 19 | KRT19 | P08727 | −3.0 | 2.0 |
| Matrix metallopeptidase 14 | MMP14 | P50281 | 1.9 | 0.2 |
| Neuregulin 1 | NRG1 | Q02297 | 1.8 | 0.2 |
| Neurofibromin 1 | NF1 | P21359 | 3.9 | 3.2 |
| Secreted phosphoprotein 1 | OPN | P10451 | 8.6 | 9.7 |
| (osteopontin) | | | | |
| Small proline-rich protein 1B | SPRR1B | P22528 | 2.4 | 1.3 |
| (cornifin) | | | | |
| Superoxide dismutase 2, | SoD | P04179 | 3.1 | 1.6 |
| mitochondrial | | | | |
| Synuclein, gamma | SNCG | O76070 | 1.9 | 0.6 |
| Telomerase RNA component | TERC | / | 2.6 | 0.5 |
| Telomerase-reverse | TERT | O14746 | 4.4 | 3.4 |
| transcriptase | | | | |
| Thrombospondin 1 | TSP1 | P07996 | 5.8 | 3.4 |
| Thromboxane-A2-receptor | TBXA2R | P21731 | 2.2 | 0.8 |
| Transcription factor AP-2 | TFAP2B | Q92481 | 2.9 | 1.5 |
| beta | | | | |
| Tumor necrosis factor | TNFRSF1B | P20333 | 2.7 | 1.6 |
| receptor superfamily, | | | | |
| member 1B | | | | |
| Tumor protein p53 | p53 | P04637 | 4.1 | 2.6 |

### Example 14

### Effect of Purified Laminaran Extract on Telomerase Activity

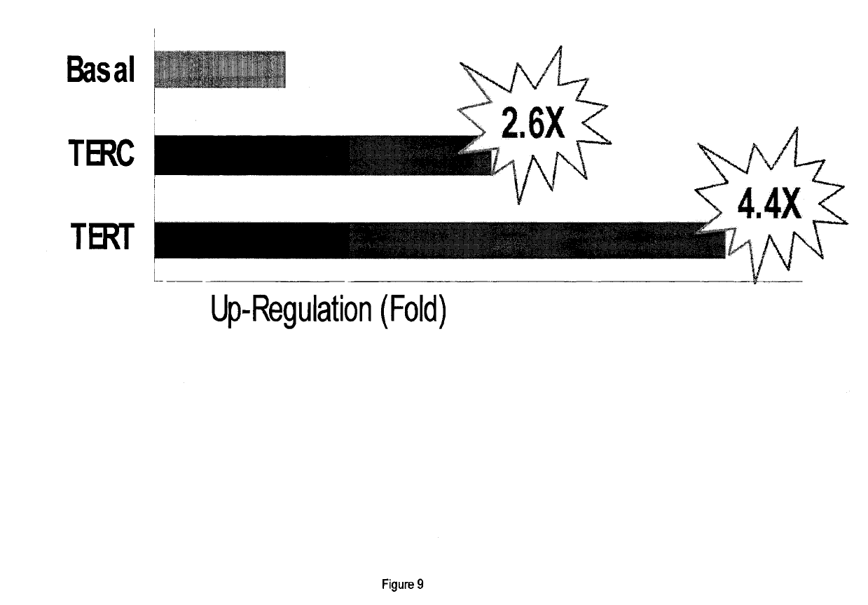

As shown in Example 13 above, both TERT and TERC were up-regulated by the treatment with the purified laminaran extract with an average ratio of 4.4 and 2.6 respectively (see FIG. 9 and Table 5 above). Their presence was unpredicted because they are not usually expressed in normal fibroblast cells. These results suggest that human dermal fibroblast treated with laminaran could extend telomere length to ultimately prevent aging. TERC serves as a template fort the TTAGGG synthesis and TERT utilises the integrated RNA molecule for DNA synthesis (De Boeck et al. 2009).

### Example 15

### Effect of Purified Laminaran Extract on Matrix Metalloproteinases Specific Activity and Collagen-I Secretion

### Quantitative Determination of Collagen Type I Secretion

Cell culture supernatants human HDFs isolated from biopsies cultured in DMEM and 10% and FBS used at passage 6. Cells were seeded in 12-well plates at a density of 5 000 cells per well. After 24 hours, 1 well per plate was trypsinized and cells counted using Coulter counter. Other wells were then washed with medium without FBS and treated with laminaran in DMEM with 0.5% FBS. Culture media were changed every two days. They were collected 48 h after the last medium change were centrifuged to remove any particulate material, and stored at −70° C. until assessed (supernatant). At the used supernatant dilutions, the culture media were not found to interfere with any of the assays. Quantitative determination of collagen type I secretion was indirectly detected using a Procollagen Type 1 C-peptide (PIP) in vitro enzyme immunoassay (EIA) kit (Takara Bio Inc., Wisconsin, USA). The amount of the free propeptides stoichiometrically reflects the amount of secreted collagen type I molecules (Corriveau et al. 2009).

The purified laminaran extract prepared as described in Example 11 was used in doses of 1.25, 2.5 et 5 mg/mL.

### Results

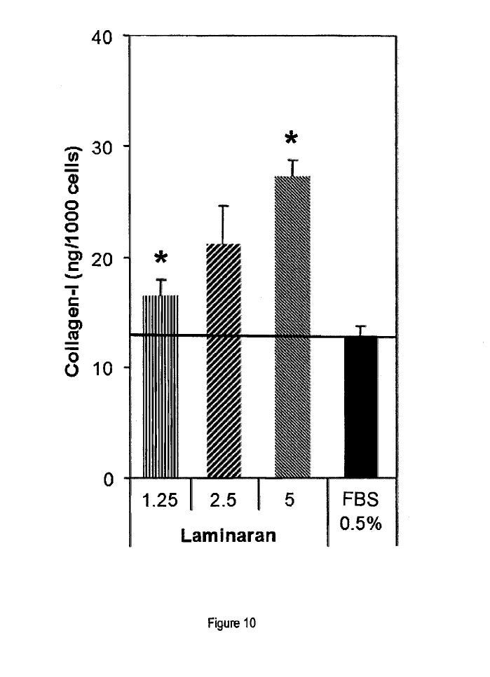

Results presented in FIG. 10 show that the purified laminaran extract increases collagen type I secretion which may help to restore extracellular matrix (ECM) integrity. The 2.5 mg/ml dose was not statistically significant because of a bias introduced by low cell viability in that group.

### Total Matrix Metalloproteinases Activity

Total matrix metalloproteinases (MMP) activity was determined in the supernatant described above as for the collagen assay using the SensoLyte™ 520 Generic MMP assay kit (Anaspec, San Jose, Calif.). This kit can detect simultaneously the activity of matrix metalloproteinases (MMP)-1, 2, 7, 8, 9, 12, 13 and 14. Results reflect the overall MMP and metalloproteinase inhibitor balance. The culture media and tested samples used were not found to interfere at the dilutions used.

The purified laminaran extract prepared as described in Example 11 was used in doses of 1.25, 2.5 et 5 mg/mL.

### Results

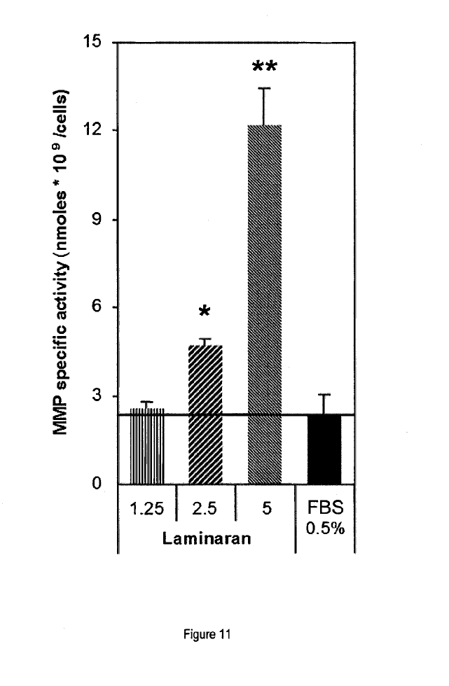

Results presented in FIG. 11 show that the purified laminaran extract increases overall MMPs activity which allows for enhanced ECM remodelling and rejuvenation of the skin. Of note, the fact that overall MMP activity is increased does not exclude that certain MMPs may advantageously be decreased.

### Statistical Analysis

All the results presented for Collagen type I secretion and MMP activity were the average±standard deviation of the results. Statistical differences were computed by the Student's t-test. The results were considered significant when P-values were <0.05 (Scherrer 1984).

### Example 16

### Effect of Crude Laminaran Extract from Laminaria digitata on Cell Growth

### Cells

Normal skin dermal fibroblasts were isolated from human skin biopsies as described previously (Moulin et al. 2002). All procedures involving patients were reviewed and approved by the Research Ethical Committee of the Université Laval. Cells were cultured in DMEM and 10% FBS (fetal bovine serum) (Moulin et al. 2004) and were used between passages 4 and 6.

### Treatment

A commercial extract of laminaran (Sigma, #L-9634, lot 112K3781, from Laminaria digitata) were solubilized in DME+10% SVF and sterilized using 0.22 μm filter. The concentrations tested were 5, 2.5 and 1.25 mg/ml for the commercial extract.

### Cell Treatment

The cells were seeded in 6-wells plates (BDBiosciences, Mississauga, ON, Canada) at a density of 50 000 cells per well. After 24 h, cells were treated with different doses of extract (2 ml/well). Treatments were renewed every 48 h during 6 days. The cells were then trypsinized, counted using a Coulter counter and 100 000 cells were seeded on a 25 cm² flask. After 24 h, the cells were treated with the same doses of extract that were applied during the first week and the treatments were renewed twice at intervals of 48 h. Cells were then trypsinized, counted and 10⁶ cells were washed with PBS, centrifuged and the pellets were frozen at −80° C.

### Statistical Analysis

The results presented in Table 6 below correspond to the average±standard deviation of the results. Statistical differences were computed by the Student's t-test. The results were considered significant when P-values were <0.05 (Scherrer 1984).

### Results

The growth of fibroblasts was significantly increased in the presence of the crude extract of laminaran, independently of the concentration used as shown in Table 6 below.

TABLE 6
Cell growth after 12 days of treatment with laminaran
commercial extract

| | conc (mg/ml) | | | |
|---|---|---|---|---|
| | 0 | 1.25 | 2.5 | 5 |
| Cell number/ | 373296 | 514292 | 526210 | 435916 |
| well | 357136 | 476114 | 507626 | 440966 |
| | 401172 | 526614 | 491870 | 488436 |
| mean | 377201 | 505673 | 508569 | 455106 |
| std | 22276 | 26330 | 17189 | 28975 |
| test t vs. 0 | / | 0.00297 | 0.00127 | 0.02098 |

### Example 17

### Effect of Laminariacea Laminaran Extracts on Telomerase Activity

### Cells

Normal skin dermal fibroblasts were isolated from human skin biopsies as described previously (Moulin et al. 2002). All procedures involving patients were reviewed and approved by the Research Ethical Committee of the UniversitéLaval. Cells were cultured in DMEM and 10% FBS (fetal bovine serum) (Moulin et al. 2004) and were used between passages 4 and 6.

### Treatment

A commercial extract of laminaran (Sigma, #L-9634, lot 112K3781, from Laminaria digitata) and the purified laminaran extract prepared as described in Example 11 above were solubilized in DME+10% SVF and sterilized using 0.22 μm filter. The concentrations tested were 5, 2.5 and 1.25 mg/ml for the commercial extract and 5 mg/ml for the purified laminaran extract.

### Cell Treatment

The cells were seeded in 6-wells plates (BDBiosciences, Mississauga, ON, Canada) at a density of 50 000 cells per well. After 24 h, cells were treated with different doses of extract (2 ml/well). Treatments were renewed every 48 h during 6 days. The cells were then trypsinized, counted using a Coulter counter and 100 000 cells were seeded on a 25 cm² flask. After 24 h, the cells were treated with the same doses of extract that were applied during the first week and the treatments were renewed twice at intervals of 48 h. Cells were then trypsinized, counted and 10⁶ cells were washed with PBS, centrifuged and the pellets were frozen at −80° C.

### Telomerase Activity Assay

The telomerase activity was assayed using TRAPeze® XL Telomerase detection kit from Millipore following the commercial protocol. This is a high sensitivity PCR-based method for the fluorometric detection of telomerase activity.

### Results

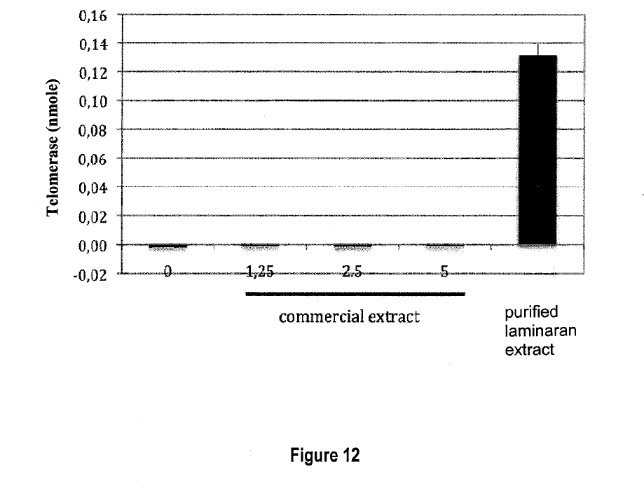

The results of the telomerase assay are presented in FIG. 12. While a strong telomerase activity was detected on fibroblasts treated with the purified laminaran extract (5 mg/ml), no telomerase activity has been detected on fibroblasts treated with the commercial laminaran extract.

### REFERENCES

1. Aubert, G., Lansdorp, P. M. (2008). Telomeres and aging. Physiological Reviews, 88(2): 557-579.
2. Bodnar, A. G., Ouellette, M., Frolkis, M., Holt, S. E., Chiu, C. P., Morin, G. B., Harley, C. B., Shay, J. W., Lichtsteiner, S., Wright, W. E. (1998). Extension of life-span by introduction of telomerase into normal human cells. Science (New York, N.Y.), 279(5349): 349-352.
3. Chapman, A. R. O. (1987). The wild harvest and culture of Laminaria longicruris in Eastern Canada in Case studies of seven commercial seaweed resources, M. S. Doty, J. F. Caddy and B. Santelices (Ed.). Rome, Food and Agriculture Organization of the United Nations. 281.
4. Corriveau, M.-P., Boufaied, I., Lessard, J., Chabaud, S., Senécal, J.-L., Grodzicky, T., Charter, S., Raymond, Y., Moulin, V. J. (2009). The fibrotic phenotype of systemic sclerosis fibroblasts varies with disease duration and severity of skin involvement: reconstitution of skin fibrosis development using a tissue engineering approach. The Journal of Pathology, Early View.
5. De Boeck, G., Forsyth, R. G., Praet, M., Hogendoom, P. C. (2009). Telomere-associated proteins: cross-talk between telomere maintenance and telomere-lengthening mechanisms. The Journal of Pathology, 217(3): 327-344.
6. de Longueville, F., Surry, D., Meneses-Lorente, G., Bertholet, V., Talbot, V., Evrard, S., Chandelier, N., Pike, A., Worboys, P., Rasson, J.-P., Le Bourdelles, B., Remade, J. (2002). Gene expression profiling of drug metabolism and toxicology markers using a low-density DNA microarray. Biochemical Pharmacology, 64(1): 137-149.
7. de Magalhäes, J. P., Chainiaux, F., de Longueville, F., Mainfroid, V., Migeot, V., Marcq, L., Remade, J., Salmon, M., Toussaint, O. (2004). Gene expression and regulation in H2O2-induced premature senescence of human foreskin fibroblasts expressing or not telomerase. Experimental Gerontology, 39(9): 1379-1389,
8. Fernandes E, Ramtani S, Coulomb B, Dubertret L, Lebreton C, Oddou C, Geiger D (Septembre 1999), Mécanique des milieux continues appliquë à la contraction d'un lattis de collagène people de fibroblasts. 14e Congrès Français de Mécanique, Toulouse.
9. Hakomori, S. I. (1964). Rapid permethylation of glycolipid, and polysaccharide catalyzed by methylsulfinyl carbanion in dimethyl sulfoxide. Journal of Biochemistry, 55(2): 205-&.
10. Hao, L. Y., Strong, M. A., Greider, C. W. (2004). Phosphorylation of H2AX at short telomeres in T cells and fibroblasts. The Journal Of Biological Chemistry, 279(43): 45148-45154.
11. Hellerqvist, C. G. (1990). Linkage analysis using lindberg method in Methods in Enzymology, J. A. McCloskey (Ed.), Academic Press. 193: 554-573.
12. Kamerling, J. P., Gerwig, G. J., Vliegenthart, J. F. G., Clamp, J. R. (1975). Characterization by gas-liquid chromatography mass spectrometry and proton-magnetic-resonance spectroscopy of pertrimethylsilyl methyl glycosides obtained in methanolysis of glycoproteins and glycopeptides. Biochemical Journal, 151(3): 491-495.
13. Larochelle, S., Langlois, C., Thibault, 1., Lopez-Valle, C. A., Roy, M., Moulin, V. (2004). Sensitivity of myofibroblasts to H2O2-mediated apoptosis and their antioxidant cell network. Journal of Cellular Physiology, 200(2): 263-271.
14. Lin, J., Jin, R., Zhang, B., Chen, H., Bai, Y. X., Yang, P. X., Han, S. W., Xie, Y. H., Huang, P. T., Huang, C., Huang, J. J. (2008). Nucleolar localization of TERT is unrelated to telomerase function in human cells. Journal of Cell Science, 121(Pt 13): 2169-2176.
15. Montreuil, J., Bouquelet, S., Debray, H., Foumet, B., Spik, G., Strecker, G. (1986). Glycoproteins in Carbohydrate analysis: a practical approach, M. F. Chaplin and J. F. Kennedy (Ed.). Washington D.C., IRL Press: 143-204.
16. Moulin, V., Auger, F. A., OConnorMcCourt, M., Germain, L. (1997). Fetal and postnatal sera differentially modulate human dermal fibroblast phenotypic and functional features in vitro. Journal of Cellular Physiology, 171(1): 1-10.
17. Moulin, V., Larochelle, S., Langlois, C., Thibault, I., Lopez-Valle, C. A., Roy, M. (2004). Normal skin wound and hypertrophic scar myofibroblasts have differential responses to apoptotic inductors. Journal of Cellular Physiology, 198(3): 350-358.
18. Moulin, V., Plamondon, M. (2002). Differential expression of collagen integrin receptor on fetal vs. adult skin fibroblasts: implication in wound contraction during healing. British Journal of Dermatology, 147(5): 886-892.
19. Nelson, T. E., Lewis, B. A. (1974). Separation and characterization of soluble and insoluble components of insoluble laminaran. Carbohydrate Research, 33(1): 63-74.
20. Nishino, T., Yokoyama, G., Dobashi, K., Fujihara, M., Nagumo, T. (1989), Isolation, purification, and characterization of fucose-containing sulfated polysaccharides from the brown seaweed Ecklonia kurome and their blood-anticoagulant activities. Carbohydrate Research, 186(1): 119-129.
21. Read, S. M., Currie, G., Bacic, A. (1996). Analysis of the structural heterogeneity of laminarin by electrospray-ionisation-mass spectrometry. Carbohydrate Research, 281(2): 187-201.
22. Scherrer, B. (1984). Biostatistique. Montreal, Paris, Morin, G., 850 p.
23. Verdun, R. E., Karlseder, J. (2007). Replication and protection of telomeres. Nature, 447(7147): 924-931.
24. Waeghe, T. J., Darvill, A. G., McNeil, M., Albersheim, P. (1983). Determination, by methylation analysis, of the glycosyl-linkage compositions of microgram quantities of complex carbohydrates. Carbohydrate Research, 123(2): 281-304.

1. A skin care composition comprising a Laminariacea extract.

2. The composition of claim 1, further comprising a topically acceptable carrier.

3. The composition of claim 1, wherein the laminariacea is Saccharina Longicruris.

4. The composition of claim 1, wherein the laminariacea is Laminaria digitata.

5. The composition of claim 1, wherein the extract is a crude Laminariacea extract.

6. The composition of claim 1, wherein the extract is a purified laminaran extract.

7. The composition of claim 1, wherein the extract is a water extract.

8. The composition of claim 7, wherein the purified extract is obtained following a first water extraction followed by an ethanol extraction.

9. The composition of claim 6, wherein the extract is substantially free of fucoidan.

10. The composition of claim 6, wherein the extract is substantially free of polyphenols.

11. The composition of claim 1, wherein the extract comprises laminaran molecules having a molecular weight of about 2000 to 9000 Da.

12. The composition of claim 1, wherein the extract comprises laminaran molecules having a molecular weight of about 2000 to about 6000 Da.

13. The composition of claim 1, wherein the extract comprises laminaran molecules having a size between about 2000 m/z and about 9000 m/z.

14. The composition of 1, wherein the extract comprises laminaran molecules having a size between about 4100 m/z and about 5800 m/z.

15. The composition of claim 1, wherein the extract comprises about 68% of D-glucose and about 1.2% of D-mannitol.

16. The composition of claim 1, wherein the extract comprises no more than one branching per chain of laminaran.

17. The composition of claim 1, wherein the branching comprises O-6 branching and O-2 branching.

18. The composition of claim 1, wherein the O-6 branching and O-2 branching are in a proportion of about 2% and about 5%, respectively.

19. The composition of claim 1, wherein the extract comprises laminaran terminated by D-mannitol and laminaran terminated by D-glucose residues.

20. The composition of claim 19, wherein the laminaran terminated by D-mannitol residues and laminaran terminated by D-glucose residues are present in a ratio of 1:1 respectively.

21. The composition of claim 1, wherein the extract is substantially free of molecules above about 10 kDa.

22. The composition of claim 1, wherein the extract is substantially free of molecules below about 1 kDa.

23. The composition of claim 1, wherein the extract is present at a concentration between about 0.01% and about 5% w/w of the composition.

24. The composition of claim 1, wherein the extract is present at a concentration between about 1.25 to about 5 mg/ml of the composition.

25. The composition of claim 1, wherein the extract is freeze-dried.

26. The composition of claim 1, wherein the extract (a) modulates the expression of a gene involved in cellular senescence; (b) modulates the expression of one or more gene(s) involved in extra cellular matrix (ECM) structure; (c) modulates the expression of one or more gene(s) involved in apoptosis; (d) modulates the expression of one or more gene(s) involved in anti-radical defences; (e) modulates the expression of one or more gene(s) involved in anti-radical differentiation; (f) improves collagen fibres thickness; (g) increases collagen tensing effect; (h) increases the expression of collagen type I; (i) increases cellular energy; (j) increases cellular differentiation; (k) increases transglutaminase K activity; (l) reduces crow's feet depth; (m) reduces mouth wrinkles; (n) increases skin firmness; (o) increases skin moisture; (p) improves skin texture; (q) improves skin microrelief; (r) promotes skin repair functions; (s) increases fibroblasts growth; (t) increases the expression of one or more gene(s) involved in telomerase activity; and/or (u) increases telomerase activity.

27. The composition of claim 1, wherein the composition (a) modulates the expression of one or more gene(s) involved in ECM structure; (b) modulates the expression of one or more gene(s) involved in apoptosis; (c) modulates the expression of one or more gene(s) involved in anti-radical defences; (d) increases collagen tensing effect; (e) increases cellular energy; (f) increases cellular differentiation; (g) increases fibroblasts growth; (h) increases the expression of one or more gene(s) involved in telomerase activity; and/or (i) increases telomerase activity.

28. The composition of claim 1, wherein the composition (a) increases the expression of at least one of BCL2-like 1, Colony stimulating factor 2, Cyclin F, Dermapontin, Fibromodulin, Heat shock 70 kDa protein 4, Insulin-like growth factor binding protein 5, Intercellular adhesion molecule 1, interleukin 11, Interleukin 8, Matrix metalloprotease 14 (MMP14), Neuregulin 1, Neurofibromin 1, osteopontin, comifin, mitochondrial Superoxide Dismutase 2, gamma synuclein, Telomerase RNA component, Telomerase reverse transcriptase, Thrombospondin 1, Thromboxane A2 receptor, Transcription factor AP-2 beta, Tumor necrosis factor receptor superfamily member 1B and Tumor protein p53; and/or (b) decreases the expression of at least one of Keratin 10 and Keratin 19.

29. The composition of claim 1, wherein the composition prevents senescence.

30. The composition of claim 1, wherein the composition extends the life span of cells.

31. The composition of claim 1, wherein the composition enhances collagen tensing effect.

32. The composition of claim 1, wherein the extract modulates apoptosis, increases anti-radical defences, increases anti-radical differentiation and/or improves the extracellular matrix of skin cells.

33. The composition of claim 1, wherein the extract increases collagen Type-I secretion.

34. The composition of claim 1, wherein the extract reduces the expression of one or more gene(s) involved in apoptosis, and wherein the one or more gene(s) involved in apoptosis are at least one of BCL2, survivin and TRAIL.

35. The composition of claim 1, wherein the extract increases the expression of one or more gene(s) involved in anti-radical defences, and wherein the one or more gene(s) involved in anti-radical defences are at least one of glutathion peroxidase, glutathion reductase, heme oxygenase and catalase.

36. The composition of claim 1, wherein the extract (a) increase the expression of one or more gene(s) involved in extra cellular matrix (ECM) structure, and wherein the one or more gene(s) involved in (ECM) optimization properties are at least one of collagen, decorin, biglycan, fibrillin, tenascin, integrin β1 and plasminogen activator inhibitor 2 (PAI 2), or (b) decreases the expression of one or more gene(s) involved in extra cellular matrix (ECM) structure, and wherein the one or more gene(s) involved in (ECM) optimization properties are at least one of metalloproteinase 1 (MMP1) and metalloproteinase 3 (MMP3).

37. The composition of claim 1, wherein the extract increases the expression of one or more gene(s) involved in anti-radical differentiation, and wherein the one or more gene(s) involved in anti-radical differentiation includes heat shock protein (HSP27).

38. The composition of claim 1, wherein the extract increases the expression of at least one of Telomerase RNA Component and Telomerase Reverse Transcriptase.

39.-53. (canceled)

54. A method for preventing or reducing a skin condition or disorder in a subject, comprising administering an effective amount of the extract defined in claim 1 or of a composition comprising this extract and a topically acceptable carrier, on the subject's skin.

55. The method of claim 54, wherein the skin condition or disorder is a skin aging sign.

56.-59. (canceled)

60. A pharmaceutical and/or nutraceutical and/or dietary composition comprising the extract defined in claim 1, and a carrier.
